(19) United States
(12) Patent Application Publication
(10) Pub. No.: US 2010/0129321 A1
(43) Pub. Date: May 27, 2010

(54) DIARYL UREA FOR TREATING VIRUS INFECTIONS

(75) Inventors: Olaf Weber, Wulfrath (DE); Bernd Riedl, Wuppertal (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: BAYER HEALTHCARE LLC, TARRYTOWN, NY (US)

(21) Appl. No.: 12/086,454

(22) PCT Filed: Dec. 6, 2006
(86) PCT No.: PCT/EP2006/011690

§ 371 Date: Jan. 5, 2010

(30) Foreign Application Priority Data
Dec. 15, 2005 (EP) 05027453.9
Dec. 15, 2005 (EP) 05027455.4
Dec. 15, 2005 (EP) 05027457.0
Dec. 15, 2005 (EP) 05027459.6
Dec. 15, 2005 (EP) 05027461.2
Dec. 15, 2005 (EP) 05027463.8
Dec. 15, 2005 (EP) 05027464.6
Dec. 15, 2005 (EP) 05027466.1
Dec. 15, 2005 (EP) 05027470.3
Dec. 15, 2005 (EP) 05027472.9

(51) Int. Cl. A61K 31/44; C07D 213/81; A61K 31/513; A61K 31/662; A61K 38/16; A61K 31/551; A61K 31/4745; A61K 31/52; A61K 31/522; A61K 31/675; A61K 31/4439; A61K 31/536; A61K 31/472; A61K 31/4725; A61K 31/496; A61K 31/7072; A61K 31/7068; A61K 31/708; A61K 35/76; A61K 36/71; A61K 38/14; A61K 38/21; A61K 31/56; A61P 31/12

(52) U.S. Cl. 424/85.6; 546/298; 514/350; 514/120; 514/12; 514/220; 514/293; 514/263.4; 514/263.38; 514/86; 514/81; 514/341; 514/274; 514/230.5; 514/307; 514/253.01; 514/253.09; 514/272; 514/50; 514/49; 514/46; 424/93.6; 424/773; 514/8; 424/85.7; 514/171

(57) ABSTRACT

The present invention relates to pharmaceutical compositions for treating virus infections and/or diseases caused thereby comprising 4{4-[3-(4-chloro-3-trifluoromethylphenyl)-ureido]-3-fluorophenoxy}-pyridine-2-carboxylic acid methylamide optionally combined with at least one additional therapeutic agent.

The present invention relates to pharmaceutical compositions for treating virus infections and/or diseases caused thereby comprising 4{4-[3-(4-chloro-3-trifluoromethylphenyl)-ureido]-3-fluorophenoxy}-pyridine-2-carboxylic acid methylamide optionally combined with at least one additional therapeutic agent.

Diaryl urea compounds e.g. 4{4-[3-(4-chloro-3-trifluoromethylphenyl)-ureido]-3-fluorophenoxy}-pyridine-2-carboxylic acid methylamide as described e.g. in US 20050038080 are potent anti-cancer and anti-angiogenic agents that possess various activities, including inhibitory activity on the VEGFR, PDGFR, raf, p38, and/or flt-3 kinase signaling molecules. These diaryl urea compounds have been previously characterized as having various activities, including for inhibiting the Raf/MEK/ERK pathway, raf kinase, p38 kinase, VEGFR kinase, PDGFR kinase. These activities and their use in treating various diseases and conditions are disclosed in, e.g., WO 2005/009961.

SARS (severe acute respiratory syndrome) is a disease caused by an infection with SARS coronavirus (SARS-CoV) which gets public importance in the last years. For infected patients the therapeutic standard of today is, however, low.

A typical coronavirus is represented by e.g. the mouse hepatitis virus (MHV) which induces the p38 kinase which is part of the MAPK pathway in infected cells (S. Banerjee et al. J. Virol. 2002, 76, 5937-5948). Furthermore recent results show that also SARS-CoV induces the signal pathway of p38 MAPK in permissive cells (Mizutani et al. Biochem. Biophys. Res. Commun. 2004, 319, 1228-1234).

A known standard therapy of HIV (human immunodeficiency virus) infections is HAART (highly active antiretroviral therapy) wherein a combination of several antiretroviral drugs (protease inhibitors and antiretroviral drugs) are administered to infected patients (e.g. a combination of indinavir, zidovudine and lamivudin). The drugs inhibit the ability of the virus to multiply in the body and slow the development of AIDS (acquired immunodeficiency syndrome).

Furthermore it is known that the p38 kinase inhibitor RWJ 67657 suppresses the replication of HIV and the cellular pathogenesis of the infection (K. Muthumani et al. AIDS, 2004, 18, 739-748).

Hepatitis viruses such as HBV and HCV modulate the MAPK signal pathway in infected cells (M. Panteva et al. Virus Research 2003, 92, 131). A permanent activation of the RAF/MEK/ERK signal pathway is detected in cells expressing HCV Core Protein (S. Giambartolomei et al., Oncogene, 2001, 20, 2607) and an increased level of N-Ras is important for the maintenance of the replication of HCV (P. Mannova, L. Beretta, J. Virol. 2005, 79 (14), 8742) wherein Ras is affected by Raf. It is also known that the integrity of the RAF/MEK signal cascade is a precondition for the replication of HBV (L. Stockl, Oncogene, 2003, 22 (17), 260).

Influenza viruses such as type A, B or C belong to group of Orthomyxoviruses and cause every year flu epidemics effecting up to 10,000 cases of death per year in Germany. Relevant cellular targets for a therapy are known (S. Ludwig et al., Trends Mol. Med., 2003, 2, 46). The p38 MAPK signal pathway is induced in mouse cells infected with influenza A virus (I. Mori et al., J. Gen. Virol. 2003, 84, 2401). Furthermore inhibition of MEK inhibit the proliferation of influenza V virus in cell cultures (S. Ludwig et al. FEBS Letters, 2004, 561, 37).

The viruses of the Herpesviridae family comprise viruses of the sub-families Alphaherpesviridae (e.g. simplexviruses such as human herpes simplex viruses and varicelloviruses such as human varizella zoster virus), Betaherpesviridae (e.g. cytomegalovirus and roseolovirus) and Gamma-herpesviridae (e.g. Epstein-Barr virus). Such virus infections can cause e.g. infections of the lymphatic system of the outer genitalia, the lips, the brian (herpesencephalitis) or the peripheral nerves.

A number of herpeviruses use the cellular signal pathways of MAPK/ERK and p38 MAPK, e.g. infection with herpes simplex virus induce the activation of the p38 MAPK and SAPK/JNK signal pathway (G. Zachos et al., J. Biol. Chem. 1999, 274, 5097). Inhibitors of the MAPK/ERK or the p38 MAPK pathway inhibit the activation of early promoters of the human cytomegalovirus (J. Chen et al. J. Virol., 2002, 76 (10), 4873).

The viruses of the Papovaviridae family comprise the genus papillomaviruses and include a “high risk” group of viruses (e.g. species HPV 16, 18) and a “low risk” group (e.g. HPV 6, 11). Human papillomaviruses induce neoplasm of the dermis and can cause the formation of papillomas. Virus infections of the “low risk” group, however, are associated with malignant tumour diseases (e.g. zervix cancer). Types of the “low risk” group cause e.g. anogenital warts. An activation of the MAPK signal pathway is detected in human papillomas infected with papillomaviruses (D. Johnston et al., Cancer Res., 1999, 59 (4), 968).

Pox were one of the most dreaded diseases in history and deemd to be exterminated in 1977 after introduction of immunisation. Today poxviruses such as the molluscum contagiosum virus and poxviruses pathogenic for animals play a role. The viruses of the Poxyiridae family include the sub-family Chordopoxyiridae and comprise avipoxvirus, capripoxvirus, lepripoxvirus, suipoxvirus, parapoxvirus, molluscipoxvirus and orthopoxvirus. Such virus infections can cause e.g. smallpox.

Cellular targets are known for the therapy of poxvirus infections (H. Yang et al., J. Clin. Invest, 2005, 115 (2), 379).

The genus flavivirus and pestivirus especially the yellow fever virus, denguevirus 1 to 4, west nile fever virus, spring-summer encephalitis virus, Omsk-hemorrhagic fever virus, bovine virus-diarrhea-virus and swine fever virus, belong to the Flaviviridae family. Such virus infections can cause e.g. encephalitis and encephalomyelitis.

Activation of the p38 MAPK signal pathway plays an important role for the interaction of Flaviviridae viruses and the host cells (C. Chen et al., J. Gen. Virol. 2002, 83, 1897).

The genus enterovirus, cardiovirus, rhinovirus, aphtovirus and hepatovirus especially the polioviruses, coxsackieviruses, coxsackieviruses, human echoviruses, human enteroviruses, human rhinoviruses and hanks viruses, belong to the Picornaviridae family. Such virus infections can cause e.g. in humans aseptic meningitis, poliomyelitis, herpangina, pleurodynia (Bornholm disease), myositis, rhabdomyolysis, diabetes type I, summer fever and myocarditis. Furthermore in animals rhinoviruses, and the foot and mouth disease viruses can be caused by such infections.

It is shown that inhibition of p38 MAPK can inhibit the replication of Picornaviridae viruses (K. Hirasawa et al., J. Virol. 2003, 77 (10), 5649-5656).

The present invention provides pharmaceutical compositions for treating virus infections and/or diseases caused thereby comprising at least one compound of formula I and optionally at least one further therapeutic agent.

The present invention provides a therapeutic method which treat virus infections according to the present invention and/or diseases caused by such infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating SARS-CoV infections and/or SARS itself comprising a compound of formula I and optionally at least one further therapeutic agent.

The present invention provides a therapeutic method which treat SARS-CoV infections and/or SARS itself of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating HIV infections and/or diseases caused by HIV infections comprising at least one compound of formula I and optionally at least one further therapeutic agent.

The present invention provides a therapeutic method which treat HIV infections and/or diseases caused by HIV infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating hepatitis virus infections and/or diseases caused by hepatitis virus infections comprising at least one compound of formula I and optionally at least one further therapeutic agent.

The present invention provides a therapeutic method which treat hepatitis virus infections and/or diseases caused by hepatitis virus infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating influenza virus infections and/or diseases caused by influenza virus infections comprising at least one compound of formula I and optionally at least one further therapeutic agent.

The present invention provides a therapeutic method which treat influenza virus infections and/or diseases caused by influenza virus infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating infections by viruses of the Herpesviridae family (Herpesviridae viruses infections) and/or diseases caused by such infections comprising at least one compound of formula I and optionally at least one further therapeutic agent.

The present invention provides a therapeutic method which treat Herpesviridae viruses infections and/or diseases caused by such infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating infections by viruses of the Papovaviridae family (Papovaviridae viruses infections) and/or diseases caused by such infections comprising at least one compound of formula I and optionally at least one further therapeutic agent.

The present invention provides a therapeutic method which treat Papovaviridae viruses infections and/or diseases caused by such infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating infections by viruses of families selected from the group consisting of Reoviridae, Astroviridae, Bunyaviridae, Filoviridae, Arenaviridae, Rhabdoviridae, Togaviridae, Paramyxoviridae and unclassified prions and/or diseases caused by such infections comprising at least one compound of formula I and optionally at least one further therapeutic agent.

The present invention provides pharmaceutical compositions for treating infections by viruses of the Poxyiridae family (Poxyiridae viruses infections) and/or diseases caused by such infections comprising at least one compound of formula I and optionally at least one further therapeutic agent.

The present invention provides a therapeutic method which treat Poxyiridae viruses infections and/or diseases caused by such infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating infections by viruses of the Flaviviridae family (Flaviviridae viruses infections) and/or diseases caused by such infections comprising at least one compound of formula I and optionally at least one further therapeutic agent.

The present invention provides a therapeutic method which treat Flaviviridae viruses infections and/or diseases caused by such infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating infections by viruses of the Picornaviridae family (Picornaviridae viruses infections) and/or diseases caused by such infections comprising at least one compound of formula I and optionally at least one further therapeutic agent.

The present invention provides a therapeutic method which treat Picornaviridae viruses infections and/or diseases caused by such infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The compounds with the structure of formula I, pharmaceutically acceptable salts, polymorphs, solvates, hydrates metabolites and prodrugs thereof, including diastereoisomeric forms (both isolated stereoisomers and mixtures of stereoisomers) are collectively referred to herein as the “compounds of formula I”.

Formula (I) is as follows:

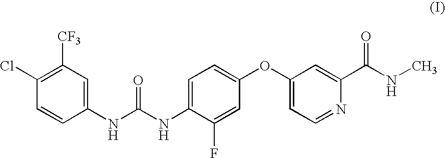

Where the plural form of the word compounds, salts, and the like, is used herein, this is taken to mean also a single compound, salt, or the like.

The present invention also relates to useful forms of the compounds as disclosed herein, such as pharmaceutically acceptable salts, metabolites and prodrugs. The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19. Pharmaceutically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, mangnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts are prepared by reacting the compounds of the invention with the appropriate base via a variety of known methods.

Representative salts of the compounds of this invention include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, trifluoromethanesulfonate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aryl or aralkyl halides like benzyl and phenethyl bromides and others monosubstituted aralkyl halides or polysubstituted aralkyl halides.

Solvates for the purposes of the invention are those forms of the compounds where solvent molecules form a complex in the solid state and include, but are not limited to for example ethanol and methanol. Hydrates are a specific form of solvates, where the solvent molecule is water.

Certain pharmacologically active agents can be further modified with labile functional groups that are cleaved after in vivo administration to furnish the parent active agent and the pharmacologically inactive derivatizing group. These derivatives, commonly referred to as prodrugs, can be used, for example, to alter the physicochemical properties of the active agent, to target the active agent to a specific tissue, to alter the pharmacokinetic and pharmacodynamic properties of the active agent, and to reduce undesirable side effects. Prodrugs of the invention include, e.g., the esters of appropriate compounds of this invention that are well-tolerated, pharmaceutically acceptable esters such as alkyl esters including methyl, ethyl, propyl, isopropyl, butyl, isobutyl or pentyl esters. Additional esters such as phenyl-C₁-C₅ alkyl may be used, although methyl ester is preferred.

Methods which can be used to synthesize other prodrugs are described in the following reviews on the subject, which are incorporated herein by reference for their description of these synthesis methods:
Higuchi, T.; Stella, V. eds. Prodrugs As Novel Drug Delivery Systems. ACS Symposium Series. American Chemical Society: Washington, D.C. (1975).
Roche, E. B. Design of Biopharmaceutical Properties through Prodrugs and Analogs. American Pharmaceutical Association: Washington, D.C. (1977).
Sinkula, A. A.; Yalkowsky, S. H. J Pharm Sci. 1975, 64, 181-210.
Stella, V. J.; Charman, W. N. Naringrekar, V. H. Drugs 1985, 29, 455-473.
Bundgaard, H., ed. Design of Prodrugs. Elsevier: New York (1985).
Stella, V. J.; Himmelstein, K. J. J. Med. Chem. 1980, 23, 1275-1282.
Han, H-K; Amidon, G. L. AAPS Pharmsci 2000, 2, 1-11.
Denny, W. A. Eur. J. Med. Chem. 2001, 36, 577-595.
Wermuth, C. G. in Wermuth, C. G. ed. The Practice of Medicinal Chemistry Academic Press: San Diego (1996), 697-715.
Balant, L. P.; Doelker, E. in Wolff, M. E. ed. Burgers Medicinal Chemistry And Drug Discovery John Wiley & Sons: New York (1997), 949-982.

The metabolites of the compounds of this invention include oxidized derivatives of the compounds of formula I, wherein one or more of the nitrogens are substituted with a hydroxy group; which includes derivatives where the nitrogen atom of the pyridine group is in the oxide form, referred to in the art as 1-oxo-pyridine or has a hydroxy substituent, referred to in the art as 1-hydroxypyridine.

### General Preparative Methods

The compounds of the invention may be prepared by use of known chemical reactions and procedures as described e.g. in the following published international application WO 2005/009961.

### Further Therapeutic Agents

The compounds of formula I according to the present invention can be combined with further therapeutic agents such as anti-viral agents, corticosteroids, immunomodulatory agents and/or known drugs for the therapy of SARS coronavirus infections and/or SARS itself.

Examples of anti-viral agents include, but are not limited to, e.g. ribavirin, lopinavir, ritonavir, the combination of lopinavir and ritonavir (Kaletra), AG 7088, hexapeptidyl CMK, interferon-β, interferon alfacon-1, interferon-α and pegylated interferon-α. Preference as further therapeutic agent is given to lopinavir and/or ritonavir.

Examples of corticosteroids include, but are not limited to, e.g. aldosteron, hydrocortisone, dexamethasone, prednisolone, methylprednisolone and cortisol.

Examples of immunomodulatory agents include, but are not limited to, e.g immunoglobulin, convalescent plasma, interferon-β, interferon alfacon-1, interferon-α and pegylated interferon-α.

The compounds of formula I according to the present invention can be combined with further therapeutic agents such as antiviral, antiretroviral agents, immunomodulatory agents and/or known drugs for the therapy of HIV infections and/or diseases caused by HIV infections.

Examples of antiviral or antiretroviral agents include, but are not limited to, e.g. lamivudin (3TC), abacavir, tenofovir disproxil fumarat, emtricitabine, didanosine, stavudine, zidovudine, zalcitabine, efavirenz, nivirapine, delaviridine, atazanavir, ritonavir, amprenavir, lopinavir, rironavir, nelfinavir, indinavir, saquinavir, enfuvirtide, etravirine, capravirine and tenofovir. Preference is given to indinavir, zidovudine, tenofovir, parapoxvirus ovis and lamivudin.

Examples of immunomodulatory agents include, but are not limited to, e.g. parapoxvirus ovis.

The compounds of formula I according to the present invention can be combined with further therapeutic agents such as anti-viral agents and/or immunomodulatory agents.

Examples of anti-viral agents include, but are not limited to, e.g. lamivudin (3TC), ribavirin, adevovir, adevovir dipivoxil, entecavir, emtricitabine, clevudine, L-dT, L-Fd4C, interferon-α and pegylated interferon-α. Preference as further therapeutic agent is given to lamivudin and/or adevovir dipivoxil.

Examples of immunomodulatory agents include, but are not limited to, e.g. parapoxvirus ovis, CpG-oligonucleotide, thymosin-α, interferon-α and pegylated interferon-α. Preference as immunomodulatory agent is given to pegylated interferon-α.

The compounds of formula I according to the present invention can be combined with further therapeutic agents such as anti-viral agents and/or immunomodulatory agents.

Examples of anti-viral agents include, but are not limited to, e.g. amantidin, symmetrel, flumadine, oseltamvir and zanamivir. Preference is given to oseltamvir and zanamivir.

Examples of immunomodulatory agents include, but are not limited to, e.g. parapoxvirus ovis, interferon-β, interferon alfacon-1, interferon-α and pegylated interferon-α. Preference as immunomodulatory agent is given to pegylated interferon-α.

The compounds of formula I according to the present invention can be combined with further therapeutic agents such as antiviral agents, immunomodulatory agents (e.g. immunoglobulins), antiviral antibodies, inhibitors of the helikase-primase complex and/or known drugs for the therapy of Herpesviridae viruses infections and/or diseases caused by Herpesviridae viruses infections.

Examples of antiviral agents include, but are not limited to, e.g. acyclovir, valacyclovir, peniciclovir, famicilovir, foscarnet, brivudin, ganciclovir and cidofovir. Preference is given to acyclovir.

The compounds of formula I according to the present invention can be combined with further therapeutic agents such as antiviral agents, immunomodulatory agents, vaccines and/or known drugs for the therapy of Papovaviridae viruses infections and/or diseases caused by Papovaviridae viruses infections.

Examples of further therapeutic agents include, but are not limited to, e.g. interferon, imiquimod, resiquimod, podophyllin, bleomycin and retinoid.

Furthermore compounds and combinations of the present invention can be used in combination with a laser therapy, a photodynamic therapy or a thermo-cauterization.

The compounds of formula I according to the present invention can be combined with further therapeutic agents such as antiviral agents, immunomodulatory agents and/or known drugs for the therapy of viruses infections according to the invention and/or diseases caused by such virus infections.

Examples of antiviral and/or immunomodulatory agents include, but are not limited to, e.g. interferon-β, interferon alfacon-1, interferon-α or pegylated interferon-α.

The compounds of formula I according to the present invention can be combined with further therapeutic agents such as antiviral agents, corticosteroids, immunomodulatory agents and/or known drugs for the therapy of Poxyiridae viruses infections and/or diseases caused by Poxyiridae viruses infections.

Examples of antiviral and/or immunomodulatory agents include, but are not limited to, e.g. cidofovir, interferon-β, interferon alfacon-1, interferon-α or pegylated interferon-α.

The compounds of formula I according to the present invention can be combined with further therapeutic agents such as antiviral agents, corticosteroids, immunomodulatory agents and/or known drugs for the therapy of Flaviviridae viruses infections and/or diseases caused by Flaviviridae viruses infections.

Examples of antiviral and/or immunomodulatory agents include, but are not limited to, e.g. ribavirin, interferon-β, interferon alfacon-1, interferon-α or pegylated interferon-α.

The compounds of formula I according to the present invention can be combined with further therapeutic agents such as antiviral agents, immunomodulatory agents and/or known drugs for the therapy of Picornaviridae viruses infections and/or diseases caused by Picornaviridae viruses infections.

Examples of antiviral agents include, but are not limited to, e.g. ruprintrivir (AG 7088), 3C protease inhibitors, pirodavir, pleconaril and soluble ICAM-1. Preference is given to ruprintrivir and pirodavir.

Examples of immunomodulatory agents include, but are not limited to, e.g. parapoxvirus ovis, interferon-β, interferon alfacon-1, interferon-α or pegylated interferon-α. Preference is given to parapoxvirus ovis and pegylated interferon-α.

### Indications

The compounds and combinations according to the present invention can be used for manufacture of a medicament for treating SARS-CoV infections and/or SARS itself. Also the present invention provides methods of treating SARS-CoV infections and/or SARS itself comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., invertebrates, vertebrates, mammals (e.g., humans; non-human primates; monkeys; livestock, such as cows, pigs, and sheep; dogs; cats; rodents; rats; mice), and birds (e.g., chicken; turkey; and ducks).

Treatment of the virus infections and diseases caused or associated with such infections according to the invention include not only the treatment of subjects who are infected by the virus, but also the treatment of subjects in which the infection or disease has not yet appeared, become symptomatic, or erupted. The present invention further relates to preventing or reducing recurring eruptions or attacks associated with viral infection. The term “treating” is used conventionally, e.g., the management or care of a subject for the purpose of combating, alleviating, reducing, relieving, improving, etc., one or more symptoms of the viral infection or associated disease.

Furthermore compounds and combinations according to the invention inhibit replication of SARS-CoV and show further positive therapeutic effects. Also compounds and combinations according to the present invention can be used for treating SARS infections with coronavirus lines which are resistant to standard therapies.

Any symptom of SARS-CoV can be treated in accordance with the present invention, including e.g., fever (>38° C.), headache, dry cough, pneumonia, and/or respiratory distress.

All SARS-CoV variants can be treated in accordance with the present invention, including, but not limited to, e.g., TOR2 (AY274119); Urbani (AY278741); CUHK-W1 (AY278554); CUHK-Su10 (AY282752); HKU-39849 (AY278491); SIN2500 (AY283794); SIN2677 (AY283795); SIN2679 (AY283796); SIN2748 (AY283797); SIN2774 (AY283798); TW1 (AY291451); BJ01 (AY278488); BJ02 (AY278487); BJ03 (AY278490); BJ04 (AY279354); GZ01 (AY278489); and sequence variations and mutations of SARS-CoV, including those which increase the pathogenicity and/or transmission modes. See, also, Pavlovic-Lazetic et al., BMC Bioinformatics 2004, 5:65, e.g., Table 1; Zhao et al., BMC Evolutionary Biology 2004:21; Yeh et al., Proc. Natl. Acad. Sci., 101:2542, 2004. For example, mutations in the Spike gene have been suggested as essential for the transition from animal-to-human transmission. See, e.g., Song et al., Proc. Natl. Acad. Sci, 102:2430, 2005.

The compounds and combinations according to the present invention can be used for manufacture of a medicament for treating HIV infections and/or diseases caused by HIV infections. Also the present invention provides methods of treating HIV infections and/or diseases caused by HIV infections comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., invertebrates, vertebrates, mammals (e.g., humans; non-human primates; monkeys; livestock, such as cows, pigs, and sheep; dogs; cats; rodents; rats; mice), and birds (e.g., chicken; turkey; and ducks).

Any strain, subtype, etc., of HIV can be treated in accordance with the present invention, including viruses related to HIV. These include, but are not limited to, e.g., HIV-1 (e.g., clades A, B, C, D, F, G, R5 and R5X4 viruses, including recombinants thereof, such as A/D, etc.), HIV-2 (e.g., R5 and R5X4 viruses, etc.), simian immunodeficiency virus (SIV), simian/human immunodeficiency virus (SHIV), feline immunodeficiency virus (FIV), bovine immunodeficiency virus (BIV) (Wright et al., Vet. Res. Commun., 26:239-50, 2002), HTLV-1, HTLV-2, etc. Phylogenetic analysis has classified HIV-1 into three groups: the major (M) group, the outlier (O) group, and the non-M, non-O (N) group. Group M is responsible for the majority of HIV infections. The other two groups are highly diverse and less prevalent. Group M isolates can be subdivided into nine subtypes (A to D, F to H, J, and K) and a number of circulating recombinant forms (CRFs), which have identical mosaic genomes and are assumed to have arisen by recombination between different subtypes. In HIV-2, only types A and B have been found in any significant number of people. See, e.g., Robertson et al., Science, 2000, 288, 55; HIV database at the worldwide web (www) address hiv.lanl.gov.

Treatment of the virus infections and diseases caused or associated with such infections according to the invention include not only the treatment of subjects who are infected by the virus, but also the treatment of subjects in which the infection or disease has not yet appeared, or become symptomatic. For example, subjects can be treated who have tested positive for HIV virus (e.g., using PCR, RT-PCR, etc.), HIV antibody (e.g., gp120, gp41, gp120/160, p24, etc., antibodies), or HIV antigens, but have not manifested the disease (e.g., decling CD4 T-cell counts are considered to be a marker of the progression of HIV infection; AIDS, e.g., when the count drops below 200 cells per cubic millimeter, or when opportunistic infections occur). Subjects can also be selected for treatment with a compound of the present invention who are specific stages of the disease, e.g., having AIDS; experiencing immune collapse; having levels of CD4 T-cells below a specified value, e.g., below about 200 cells, below about 500 cells; having levels of viral load above a specified value, e.g., greater than about 5,000 copies HIV RNA per ml plasma, greater than about 5,000 copies HIV RNA per ml plasma, greater than about 5,000 copies HIV RNA per ml plasma, etc.

The present invention further relates to preventing or reducing symptoms associated with viral infection. These include symptoms associated with the minor symptomatic phase of HIV infection, including, e.g., shingles; skin rash and nail infection; mouth sores; recurrent nose and throat infection; and weight loss. In addition, further symptoms of associated with the major symptomatic phase of HIV infection, include, e.g., oral and vaginal thrush (Candida); persistent diarrhoea; weight loss; persistant cough and reactivated tuberculosis; recurrent herpes infections such as cold sores (herpes simplex), Symptoms of full-blown AIDS which can be treated in accordance with the present invention, include, e.g., diarrhoea, nausea and vomiting; thrush and mouth sores; persistent, recurrent vaginal infections and cervical cancer; persistent generalised lymphadenopathy (PGL); severe skin infections, warts and ringworm; respiratory infections; pneumonia, especially pneumocystis carinii pneumonia (PCP); herpes zoster (or shingles); nervous system problems, such as pains, numbness or “pins and needles” in the hands and feet; neurological abnormalities; Kaposi's sarcoma; lymphoma; tuberculosis, e.g., the occurrence of opportunistic infections; Karposi. The term “treating” is used conventionally, e.g., the management or care of a subject for the purpose of combating, alleviating, reducing, relieving, improving, etc., one or more symptoms of the viral infection or associated disease.

Furthermore compounds and combinations according to the invention inhibit replication of HIV and show further positive therapeutic effects. Also compounds and combinations according to the present invention can be used for treating HIV infections with virus lines which are resistant to standard therapies.

Examples of diseases caused by HIV infections include, but are not limited to, e.g. AIDS (acquired immunodeficiency syndrome) and Kaposi's syndrome.

The compounds and combinations according to the present invention can be used for manufacture of a medicament for treating hepatitis virus infections and/or diseases caused by hepatitis virus infections. Also the present invention provides methods of treating hepatitis virus infections and/or diseases caused by hepatitis virus infections comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., invertebrates, vertebrates, mammals (e.g., humans; non-human primates; monkeys; livestock, such as cows, pigs, and sheep; dogs; cats; rodents; rats; mice), and birds (e.g., chicken; turkey; and ducks).

Treatment of the virus infections and diseases caused or associated with such infections according to the invention include not only the treatment of subjects who are infected by the virus, but also the treatment of subjects in which the infection or disease has not yet appeared or become symptomatic. The present invention further relates to preventing or reducing recurring attacks associated with viral infection. The term “treating” is used conventionally, e.g., the management or care of a subject for the purpose of combating, alleviating, reducing, relieving, improving, etc., one or more symptoms of the viral infection or associated disease.

Furthermore compounds and combinations according to the invention inhibit replication of hepatitis virus infections and show further positive therapeutic effects. Also compounds and combinations according to the invention can be used for treating infections with hepatitis virus lines which are resistant to standard therapies.

Examples of hepatitis virus infections include, but are not limited to, e.g. infections with hepatitis A virus (HAV), hepatitis B virus (HBV), hepatitis C virus (HCV), hepatitis D virus (HDV), hepatitis E virus (HEV) and hepatitis G virus (HGV). Preference is given to infections with human hepatitis virus. More preferably HCV and/or HBV infections are mentioned.

Any type, strain, or species of hepatitis can be treated in accordance with the present invention, including all mammalian strains, e.g., human, porcine, etc. The main HCV genotypes include types 1, 2, 3, 4, 5, 6, 7, 8, 9, 10, and 11. These can be further classified into: 1a, 1b, 1c, 2a, 2b, 2c, 3a, 3b, 4a-4-e, 5a, 6a, 7a, 7b, 8a, 8b, 9a, 10a, and 11a. See, also, e.g., Stuyver et al. (1993), Typing of hepatitis C virus (HCV) isolates and characterization of new (sub)types using a Line Probe Assay. J Gen Virology, 74: 1093-1102; Stuyver et al. (1996), Second-generation line probe assay for hepatitis C virus genotyping. J. Clin. Microbiol. 34, 2259-2266; U.S. Patent Application Nos. 20050069870. HBV can be classified into seven strains, e.g., A-H. See, also, Miyakawa and Mizokami, Intervirology, 2003; 46(6):329-38. isolates of HEV have been classified by genomic analysis into at least types 1, 2, 3, and 4.

Examples of diseases caused by hepatitis virus infection include, but are not limited to, e.g. hepatitis, cirrhosis and cancer of the liver, jaundice, chronically infection of the liver and associated diseases and modifications of the liver thereof.

The compounds and combinations according to the present invention can be used for manufacture of a medicament for treating influenza virus infections and/or diseases caused by influenza virus infections. Also the present invention provides methods of treating influenza virus infections and/or diseases caused by influenza virus infections comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., invertebrates, vertebrates, mammals (e.g., humans; non-human primates; monkeys; livestock, such as cows, pigs, and sheep; dogs; cats; rodents; rats; mice), and birds (e.g., chicken; turkey; and ducks).

Treatment of the virus infections and diseases caused or associated with such infections according to the invention include not only the treatment of subjects who are infected by the virus, but also the treatment of subjects in which the infection or disease has not yet appeared, become symptomatic, or erupted. The present invention further relates to preventing or reducing recurring eruptions or attacks associated with viral infection. The term “treating” is used conventionally, e.g., the management or care of a subject for the purpose of combating, alleviating, reducing, relieving, improving, etc., one or more symptoms of the viral infection or associated disease.

Furthermore compounds and combinations according to the invention inhibit replication of influenza virus infections and show further positive therapeutic effects. Also compounds and combinations according to the invention can be used for treating infections with influenza virus lines which are resistant to standard therapies.

Examples of influenza virus infections include, but are not limited to, e.g. infections with ortho-myxoviruses, influenza A virus, influenza B virus and influenza C virus.

Examples of influenza viral infections that can be treated in accordance with the present invention include, e.g., influenza virus A (including all strains varying in their HA and NA proteins, such as HIN1, HIN2, and H3N2; H7N7; H3N8); influenza B, influenza C, thogoto virus (including Dhori, Batken virus, SiAR 126 virus), and isavirus (e.g., infectious salmon anemia virus). These include influenza isolated or transmitted from all species types, including isolates from invertebrates, vertebrates, mammals, humans, non-human primates, monkeys, pigs, cows, and other livestock, birds, domestic poultry such as turkeys, chickens, quail, and ducks, wild birds (including aquatic and terrestrial birds), reptiles, etc. These also include existing strains which have changed, e.g., through mutation, antigenic drift, antigenic shift, recombination, etc., especially strains which have increased virulence and/or interspecies transmission (e.g., human-to-human).

Of particular interest are influenza viruses which are panzootic and/or which cross species either because they have a broad host range, by recombination in the infected host, and/or mutation. For example, H5N1 (in reference to the subtypes of surface antigens present on the virus, hemagglutinin type 5 and neuraminadase type 1) is a subtype of avian influenza A, which caused an outbreak of flu in domestic birds in Asia. As of November 2005, more 120 million birds died from infection or were killed to prevent further infection from spreading. This virus has also spread into human hosts (“bird flu”) where it is associated with high lethality.

Avian influenza A virus strains can be classified as low pathogenic (LPAI) or highly pathogenic (HPAI) on the basis of specific molecular genetic and pathogenesis criteria that require specific testing. Most avian influenza A viruses are LPAI viruses that are usually associated with mild disease in poultry. In contrast, HPAI viruses can cause severe illness and high mortality in poultry. More recently, some HPAI viruses (e.g., H5N1) have been found to cause no illness in some poultry, such as ducks. LPAI viruses have the potential to evolve into HPAI viruses and this has been documented in some poultry outbreaks. Avian influenza A viruses of the subtypes H5 and H7, including H5N1, H7N7, and H7N3 viruses, have been associated with HPAI, and human infection with these viruses have ranged from mild (H7N3, H7N7) to severe and fatal disease (H7N7, H5N1). Human illness due to infection with LPAI viruses has been documented, including very mild symptoms (e.g., conjunctivitis) to influenza-like illness. Examples of LPAI viruses that have infected humans include H7N7, H9N2, and H7N2. Compounds of the present invention can be utilized to treat infections associated with such viruses.

### Influenza A H5

At least nine subtypes of H5 have been identified. 1-15 infections, such as HPAI H5N1 viruses currently circulating in Asia and Europe, have been documented among humans and can cause severe illness or death.

### Influenza A H7

At least nine subtypes of H7 have been identified. H7 infection in humans is rare but can occur among persons who have direct contact with infected birds. Symptoms may include conjunctivitis and/or upper respiratory symptoms. H7 viruses have been associated with both LPAI (e.g., H7N2, H7N7) and HPAI (e.g., H7N3, H7N7), and have caused mild to severe and fatal illness in humans. The H subtypes are epidemiologically most important, as they govern the ability of the virus to bind to and enter cells, where multiplication of the virus then occurs. The N subtypes govern the release of newly formed virus from the cells

### Influenza A H9

At least nine subtypes of H9 have been identified. Influenza A H9 has rarely been reported to infect humans. However there are reports of children exhibiting flu-like syndromes when infected with H9 strains. See, e.g., Anonymous. Influenza: Hong Kong Special Administrative Region of China. W H O Weekly Epidemiol Rec. 1999; 14:111. The present invention relates to the treatment of all avian influenza subtypes (e.g., H and N subtypes), including existing subtypes, derivatives thereof, and recombinants thereof, such as subtypes and recombinants which have the ability to spread from human-to-human. Various isolates have been characterized, especially for H5 subtypes. See, e.g., Sturm-Ramirez, J. Virol., 2004, 78, 4892-4901; Guan et al., Proc. Natl. Acad. Sci., 2004, 101, 8156-8161.

Influenza subtyping can be accomplished routinely, e.g., using PCR on genomic sequences. See, also Kessler et al., J. Clin. Microbiol., 2004, 42, 2173-2185.

Examples of diseases caused by influenza virus infection include, but are not limited to, e.g. flu, bird flu, swine flu, etc.

Compounds of the present invention can treat one or more symptoms associated with influenza infection, including, e.g., fever, cough, sore throat, sore muscles, pneumonia, respiratory failure, acute respiratory distress syndrome, conjunctivitis, and toxic-shock-like syndrome (e.g., fever, chills, vomiting, and headache). Compounds of the present invention can also reduce, block, lessen, decrease, etc., the production of cytokines associated with influenza infection, e.g., reducing the occurrence of hypercytokinemia (“cytokine storm”) and the symptoms associated with over-expression of cytokines.

The compounds and combinations according to the present invention can be used for manufacture of a medicament for treating Herpesviridae viruses infections and/or diseases caused by such infections. Also the present invention provides methods of treating Herpesviridae viruses infections and/or diseases caused by such infections comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., mammals (e.g., humans; non-human primates; monkeys; livestock, such as cows, pigs, and sheep; dogs; cats; rodents; rats; mice), and birds (e.g., chicken; turkey; and ducks). See, also any of the subjects listed in Table 1.

Treatment of the virus infections and diseases caused or associated with such infections according to the invention include not only the treatment of subjects who are infected by the virus, but also the treatment of subjects in which the infection or disease has not yet appeared, become symptomatic, or erupted. The present invention further relates to preventing or reducing recurring eruptions or attacks associated with viral infection. The term “treating” is used conventionally, e.g., the management or care of a subject for the purpose of combating, alleviating, reducing, relieving, improving, etc., one or more symptoms of the viral infection or associated disease.

Furthermore compounds and combinations according to the invention inhibit replication of Herpesviridae viruses and show further positive therapeutic effects. Also compounds and combinations according to the present invention can be used for treating Herpesviridae viruses infections with virus lines which are resistant to standard therapies.

The virus family Herpesviridae include Alphaherpesviridae, Betaherpesviridae and Gamma-herpesviridae. Examples of Herpesviridae viruses include, but are not limited to, simplexviruses such as human herpes simplex viruses, varicelloviruses such as human varizella zoster virus, cytomegalovirus, roseolovirus, Epstein-Barr virus, equine viruses, Aujeszky's virus, suid virus, apish herpesviruses, cercophitecinem herpesviruses, ateline herpesvirus, bovine herpesviruses, feline herpesvirus and canine herpesvirus.

Examples of diseases caused by Herpesviridae viruses infections include, but are not limited to, e.g. infections of the lymphatic system of the outer genitalia, the lips (including oral herpes), the brain (herpesencephalitis) or the peripheral nerves. Other diseases and associated viruses include, e.g., cold or fever sores (e.g., herpes simplex 1), genital herpes (e.g., herpes simplex 2), chickenpox (varicella-zoster virus), shingles (varicella-zoster virus), infectious mononucleosis (Epstein-Barr virus), roseola (e.g., HHV-1V-6a and HHV-7), gingival stomatitis, herpes genitalis, herpes labialis, herpes gladiatorum, encephalitis, keratoconjunctivitis, Karposi's sarcoma (herpesvirus 8), etc. Any infection or diseases associated with Herpesviridae can be treated in accordance with the present invention, including those mentioned in Table 1.

The compounds and combinations according to the present invention can be used for manufacture of a medicament for treating Papovaviridae viruses infections and/or diseases caused by such infections. Also the present invention provides methods of treating Papovaviridae viruses infections and/or diseases caused by such infections comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., invertebrates, vertebrates, mammals (e.g., humans; non-human primates; monkeys; livestock, such as cows, pigs, and sheep; dogs; cats; rodents; rats; mice), and birds (e.g., chicken; turkey; and ducks). See, also any of the subjects listed in Table 1.

Treatment of the virus infections and diseases caused or associated with such infections according to the invention include not only the treatment of subjects who are infected by the virus, but also the treatment of subjects in which the infection or disease has not yet appeared, become symptomatic, or erupted. The present invention further relates to preventing or reducing recurring eruptions or attacks associated with viral infection. The term “treating” is used conventionally, e.g., the management or care of a subject for the purpose of combating, alleviating, reducing, relieving, improving, etc., one or more symptoms of the viral infection or associated disease.

Furthermore compounds and combinations according to the invention inhibit replication of Papovaviridae viruses and show further positive therapeutic effects. Also compounds and combinations according to the present invention can be used for treating Papovaviridae viruses infections with virus lines which are resistant to standard therapies.

The virus family Papovaviridae include, but is not limited to, e.g. papillomaviruses such as the human papillomaviruses (HPV 6, 11, 16, 18).

Examples of diseases caused by Papovaviridae viruses infections include, but are not limited to, e.g. papillomas, warts such as anogenital warts and neoplasm of the dermis caused by such infections.

Any Papovaviridae infection can be treated, including those listed in Table 2, and especially paillomaviral infections, such as the HPV types and diseases listed in Table 3. Subjects harbouring HPV viruses can be treated in accordance with the present invention, including subjects with asymptomatic infection, classical condylomata (genital warts), and subclinical infection (e.g., lesions not visible on routine inspection). HPV typing can be conducted routinely. See, e.g., Roman and Fife, Clinical Microb. Rev., 2:166-190, 1989.

There are two polyomaviruses found in humans: JC virus, which can infect the respiratory system, kidneys, or brain (e.g., causing the fatal progressive multifocal leukoencephalopathy), and BK virus, which produces a mild respiratory infection and can affect the kidneys of immunosuppressed transplant patients. An avian polyomavirus, referred to as the Budgerigar fledgling disease virus, is a frequent cause of death among caged birds. Any of these viruses and associated diseases can be treated in accordance with the present invention.

The compounds and combinations according to the present invention can be used for manufacture of a medicament for treating virus infections according to the present invention and/or diseases caused by such infections. Also the present invention provides methods of treating virus infections according to the present invention and/or diseases caused by such infections comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., invertebrates, vertebrates, mammals (e.g., humans; non-human primates; monkeys; livestock, such as cows, pigs, and sheep; dogs; cats; rodents; rats; mice), and birds (e.g., chicken; turkey; and ducks).

Treatment of the virus infections and diseases caused or associated with such infections according to the invention include not only the treatment of subjects who are infected by the virus, but also the treatment of subjects in which the infection or disease has not yet appeared or become symptomatic. The present invention further relates to preventing or reducing recurring eruptions or attacks associated with viral infection. The term “treating” is used conventionally, e.g., the management or care of a subject for the purpose of combating, alleviating, reducing, relieving, improving, etc., one or more symptoms of the viral infection or associated disease.

Furthermore compounds and combinations according to the invention inhibit replication of viruses according to the present invention and show further positive therapeutic effects. Also compounds and combinations according to the present invention can be used for treating virus infections according to the present invention with virus lines which are resistant to standard therapies.

Examples of viruses according to the present invention are viruses of the family Reoviridae such as human rotavirus, of the family Astroviridae such as astrovirus, of the family Bunyaviridae such as bunyamweravirus, California encephalitis virus, Hantaan virus, LaCrosse virus, Muerto Canyon virus, Rift Valley Fever virus, sandfly fever virus or tahyna virus, of the family Filoviridae such as ebola virus or Marburg virus, of the family Arenaviridae such as Junin virus, Lassa virus, lymphotropic choriomeningitis virus or Machupo virus, of the family Rhabdoviridae such as hydrophobia virus, Duvenhage virus, Mokola virus or vesicular stomatitis virus, of the family Togaviridae such as Chikungunya virus, Eastern Equine Encephalitis virus, Mayaro virus, O'nyong-nyong virus, ross fever virus, roseola virus or other Equine Encephalitis viruses, of the family Paramyxoviridae such as measles virus, mumps virus or parainfluenza virus and unclassified prions such as prions causing Jakob-Creutzfeld disease, BSE or Kuru and its different variants; family Parvoviridae, such as erythrovirus (e.g., B19 virus) and dependovirus (e.g., adeno-associated virus, AAV-2); family Adenoviridae, such as Mastadenovirus (e.g., human adenovirus serotypes 1047).

The compounds and combinations according to the present invention can be used for manufacture of a medicament for treating Poxyiridae viruses infections and/or diseases caused by such infections. Also the present invention provides methods of treating Poxyiridae viruses infections and/or diseases caused by such infections comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., mammals (e.g., humans; non-human primates; monkeys; livestock, such as cows, pigs, and sheep; dogs; cats; rodents; rats; mice), and birds (e.g., chicken; turkey; and ducks). See, also any of the subjects listed in Table 4.

Treatment of the virus infections and diseases caused or associated with such infections according to the invention include not only the treatment of subjects who are infected by the virus, but also the treatment of subjects in which the infection or disease has not yet appeared, become symptomatic, or erupted. The present invention further relates to preventing or reducing recurring eruptions or attacks associated with viral infection. The term “treating” is used conventionally, e.g., the management or care of a subject for the purpose of combating, alleviating, reducing, relieving, improving, etc., one or more symptoms of the viral infection or associated disease. For example, about 7-17 days after exposure to variola virus, an infected subject can begin to experience the first symptoms of smallpox disease. A compound administered during this time period, or at any point during the disease, can prevent or inhibit progression of the disease. The compounds can block, reduce, diminish, alleviate, etc., one or more symptoms of the disease, including, but not limited to, e.g., fever, malaise, head and body aches, vomiting, prodrome phase, typical or atypical rash during all its phases, hemorrhagic rash, hemorrhage, etc. These compounds can reduce the severity of the disease, as well as the degree and period during which it is contagious.

Adverse reactions and other effects of poxvirus vaccination can also be treated in accordance with the present invention, e.g., by administering an effective amount of a compound of the present invention. Adverse reactions to vaccinia vaccination include, but are not limited to, e.g., generalized vaccinia, progressive vaccinia, eczema vaccinatum, post-vaccinal encephalitis, vaccinial myocarditis and/or pericarditis, ocular vaccinia, encephalomyelitis (PVEM), fetal vaccinia, etc. Furthermore compounds and combinations according to the invention inhibit replication of Poxyiridae viruses and show further positive therapeutic effects. Also compounds and combinations according to the present invention can be used for treating Poxyiridae viruses infections with virus lines which are resistant to standard therapies.

Any poxvirus infection can be treated and/or prevented in accordance with the present invention, including, but not limited to, infections and diseases associated with orthopoxvirus, parapoxvirus, avipovirus, capripoxvirus, leporipoxvirus, suipoxvirus, molluscum contagiosum virus fowlpox, etc. Orthopoxvirus, include, e.g., buffalopox, camelpox, cowpox, monkeypox, rabbitpox, raccoon pox, tatera pox, canarypox, vaccinia, variola (smallpox), and vole pox. For other poxvirus, see e.g., Virology, Fields et al., Volume 2, Chapters 74-75, Raven Press, 1990.

Diseases that can be treated in accordance with the present invention include, e.g, smallpox (variola virus); cowpox (cowpox virus); contagious pustular dermatitis (orf virus); pseudocowpox (pseudocowpoxvirus); molluscum contagiousum (molluscum contagiosum virus); histocytomaa of head or limbs (Yaba monkey tumor virus); tanapox (tanapox virus), etc.

The compounds and combinations according to the present invention can be used for manufacture of a medicament for treating Flaviviridae viruses infections and/or diseases caused by such infections. Also the present invention provides methods of treating Flaviviridae viruses infections and/or diseases caused by such infections comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., mammals (e.g., humans; non-human primates; monkeys; livestock, such as cows, pigs, and sheep; dogs; cats; rodents; rats; mice), and birds (e.g., chicken; turkey; and ducks). See, also any of the subjects listed in Table 5.

Treatment of the virus infections and diseases caused or associated with such infections according to the invention include not only the treatment of subjects who are infected by the virus, but also the treatment of subjects in which the infection or disease has not yet appeared, become symptomatic, or erupted. The present invention further relates to preventing or reducing recurring eruptions or attacks associated with viral infection. The term “treating” is used conventionally, e.g., the management or care of a subject for the purpose of combating, alleviating, reducing, relieving, improving, etc., one or more symptoms of the viral infection or associated disease.

Furthermore compounds and combinations according to the invention inhibit replication of Flaviviridae viruses and show further positive therapeutic effects. Also compounds and combinations according to the present invention can be used for treating Flaviviridae viruses infections with virus lines which are resistant to standard therapies.

Examples of Flaviviridae viruses are the genus flavivirus and pestivirus such as yellow fever virus, denguevirus (e.g. species 1-4), west nile fever virus, spring-summer encephalitis virus, Omsk-hemorrhagic fever virus, bovine virus-diarrhea-virus and swine fever virus, and hepatitis C.

Examples of diseases caused by Flaviviridae viruses infections include, but are not limited to, e.g. encephalitis, encephalomyelitis, Dengue fever (e.g., DEN-1, 2, 3, -4), Yellow fever (e.g., hemorrhagic fever), St. Louis encephalitis, Japanese encephalitis, Murray Valley encephalitis, and West Nile, Rocio, Tick-borne encephalitis, Omsk hemorrhagic fever, Kyasanur Forest disease (e.g., hemorrhagic fever), and Powassan (encephalitis; meningoencephalitis).

The compounds and combinations according to the present invention can be used for manufacture of a medicament for treating Picornaviridae viruses infections and/or diseases caused by such infections. Also the present invention provides methods of treating Picornaviridae viruses infections and/or diseases caused by such infections comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., mammals (e.g., humans; non-human primates; monkeys; livestock, such as cows, pigs, and sheep; dogs; cats; rodents; rats; mice), and birds (e.g., chicken; turkey; and ducks). See, also any of the subjects listed in Table 6.

Treatment of the virus infections and diseases caused or associated with such infections according to the invention include not only the treatment of subjects who are infected by the virus, but also the treatment of subjects in which the infection or disease has not yet appeared, become symptomatic, or erupted. The present invention further relates to preventing or reducing recurring eruptions or attacks associated with viral infection. The term “treating” is used conventionally, e.g., the management or care of a subject for the purpose of combating, alleviating, reducing, relieving, improving, etc., one or more symptoms of the viral infection or associated disease.

Furthermore compounds and combinations according to the invention inhibit replication of Picornaviridae viruses and show further positive therapeutic effects. Also compounds and combinations according to the present invention can be used for treating Picornaviridae viruses infections with virus lines which are resistant to standard therapies.

Examples of Picornaviridae viruses are the genus enterovirus, cardiovirus, rhinovirus, aphtovirus and hepatovirus such as polioviruses (e.g. species 1, 2, 3), coxsackievirus (e.g. species A1-A22, A24), coxsackieviruses (e.g. species B1-B6), human echoviruses (e.g. species 1-7, 9, 11-27, 29-33), human enteroviruses (e.g. species 68-71), human rhinoviruses (e.g. species 1-100, 1A, 1B), hanks virus, rhinoviruses (e.g. species 1, 2), and the foot and mouth disease viruses (e.g. species O, A, C, SAT1-3, ASIA1).

Examples of diseases caused by Picornaviridae viruses infections in human include, but are not limited to, e.g. aseptic meningitis, poliomyelitis, herpangina, pleurodynia (Bornholm disease), myositis, rhabdomyolysis, diabetes type I, summer fever and myocarditis.

Examples of a picornaviridae virus and the disease associated with it, include, but are not limited to, Poliovirus (3 serotypes), e.g., polio; Coxsackie A virus (23 serotypes), e.g., herpangina (infection of oral mucosal cells); aseptic meningitis; common cold (upper respiratory tract infection); epidemic myalgia (including, pleurodynia, Bornholm disease, devil's grip); hand, foot, mouth disease (infection of epithelial cells of the skin and oral mucosa); Coxsackie B virus (6 serotypes), e.g., aseptic meningitis; epidemic myalgia (including, pleurodynia, Bornholm disease, devil's grip); myocarditis; pericarditis; Echovirus (32 serotypes), e.g., aseptic meningitis; Boston exanthem (epithelial cell infection); cerebellar ataxia; pneumonitis; Rhinovirus (113 serotypes), e.g., common cold; and Hepatitis A virus, e.g., infectious hepatitis.

### Administration

Compounds or drug combinations of the present invention can be administered in any form by any effective route, including, e.g., oral, parenteral, enteral, intravenous, intraperitoneal, topical, transdermal (e.g., using any standard patch), ophthalmic, nasally, local, non-oral, such as aerosal, inhalation, subcutaneous, intramuscular, buccal, sublingual, rectal, vaginal, intra-arterial, and intrathecal, etc. They can be administered alone, or in combination with any ingredient(s), active or inactive.

Preference is given to an oral administration.

Compounds or drug combinations of the present invention can be converted in a known manner into the usual formulations, which may be liquid or solid formulations e.g. without limitation normal and enteric coated tablets, capsules, pills, powders, granules, elixirs, tinctures, solution, suspensions, syrups, solid and liquid aerosols and emulsions.

Examples of solid formulations for oral administration are described in U.S. provisional application No. 60/605,752.

The combinations of the present invention can be administered at any time and in any effective form. For example, the compounds can be administered simultaneously, e.g., as a single composition or dosage unit (e.g., a pill or liquid containing both compositions), or they can be administered as separate compositions, but at the same time (e.g., where one drug is administered intravenously and the other is administered orally or intramuscularly). The drugs can also be administered sequentially at different times. Agents can be formulated conventionally to achieve the desired rates of release over extended period of times, e.g., 12-hours, 24-hours. This can be achieved by using agents and/or their derivatives which have suitable metabolic half-lives, and/or by using controlled release formulations.

The drug combinations can be synergistic, e.g., where the joint action of the drugs is such that the combined effect is greater than the algebraic sum of their individual effects. Thus, reduced amounts of the drugs can be administered, e.g., reducing toxicity or other deleterious or unwanted effects, and/or using the same amounts as used when the agents are administered alone, but achieving greater efficacy. The reduced amounts of the drugs can be lower then used in a standard therapy wherein e.g. the single drug is administered.

Compounds or drug combinations of the present invention can be further combined with any other suitable additive or pharmaceutically acceptable carrier. Such additives include any of the substances already mentioned, as well as any of those used conventionally, such as those described in Remington: The Science and Practice of Pharmacy (Gennaro and Gennaro, eds, 20th edition, Lippincott Williams & Wilkins, 2000); Theory and Practice of Industrial Pharmacy (Lachman et al., eds., 3rd edition, Lippincott Williams & Wilkins, 1986); Encyclopedia of Pharmaceutical Technology (Swarbrick and Boylan, eds., 2nd edition, Marcel Dekker, 2002). These can be referred to herein as “pharmaceutically acceptable carriers” to indicate they are combined with the active drug and can be administered safely to a subject for therapeutic purposes.

In addition, compounds or drug combinations of the present invention can be administered with other active agents or other therapies that are utilized to treat any of the above-mentioned diseases and/or conditions.

Other therapies according to the invention include, but are not limited to, physical or mechanical therapy such as electrical stimulation, acupuncture, magnet therapy or topical use of polyurethane films.

The present invention provides also combinations of at least one compound of Formula I and at least one other therapeutic agent mentioned above useful in treating a disease or disorder. “Combinations” for the purposes of the invention include:
single compositions or dosage forms which contain at least one compound of Formula I and at least one other therapeutic agent mentioned above;
combination packs containing at least one compound of Formula I and at least one other therapeutic agent mentioned above to be administered concurrently or sequentially;
kits which comprise at least one compound of Formula I and at least one other therapeutic agent mentioned above packaged separate from one another as unit dosages or as independent unit dosages, with or without instructions that they be administered concurrently or sequentially; and
separate independent dosage forms of at least one compound of Formula I and at least one other therapeutic agent mentioned above which cooperate to achieve a therapeutic effect, e.g., treatment of the same disease, when administered concurrently or sequentially.

The dosage of each agent of the combination can be selected with reference to the other and/or the type of disease and/or the disease status in order to provide the desired therapeutic activity. For example, the active agents in the combination can be present and administered in a fixed combination. “Fixed combination” is intended here to mean pharmaceutical forms in which the components are present in a fixed ratio that provides the desired efficacy. These amounts can be determined routinely for a particular patient, where various parameters are utilized to select the appropriate dosage (e.g., type of disease, age of patient, disease status, patient health, weight, etc.), or the amounts can be relatively standard.

The amount of the administered active ingredient can vary widely according to such considerations as the particular compound and dosage unit employed, the mode and time of administration, the period of treatment, the age, sex, and general condition of the patient treated, the nature and extent of the condition treated, the rate of drug metabolism and excretion, the potential drug combinations and drug-drug interactions, and the like.

Preference is given to an amount of the compound of formula I from 20 to 2000 mg, preferably from 40 to 800 mg, more preferably from 50 to 600 mg.

Particular preference is given to an amount of 4{4-[3-(4-chloro-3-trifluoromethylphenyl)-ureido]-3-fluorophenoxy}-pyridine-2-carboxylic acid methylamide in the pharmaceutical composition from 20 to 3000 mg, preferably from 50 to 1500, more preferably from 60 to 1000 mg.

In another embodiment of the invention the compound of formula I is administered in combination with at least one further therapeutic agent in an amount that those of ordinary skill in the art can determine by their professional judgement.

The pharmaceutical composition according to the invention is administered one or more, preferably up to three, more preferably up to two times per day. Preference is given to an administration via the oral route. With each administration the number of tablets or capsules taken in at the same time should not exceed two.

Nevertheless, it may in some cases be advantageous to deviate from the amounts specified, depending on body weight, individual behavior toward the active ingredient, type of preparation and time or interval over which the administration is effected. For instance, less than the aforementioned minimum amounts may be sufficient in some cases, while the upper limit specified has to be exceeded in other cases. In the case of administration of relatively large amounts, it may be advisable to divide these into several individual doses over the day.

The combination can comprise effective amounts of at least one compound of Formula I and at least one other therapeutic agent mentioned above, which achieves a greater therapeutic efficacy than when either compound is used alone. The combination can be useful to treat SARS-CoV infections and/or SARS itself, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating SARS-CoV infections and/or SARS itself where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising a compound of formula I and lopinavir and/or ritonavir. More preferably a combination comprising 4{4-[3-(4-chloro-3-trifluoromethylphenyl)-ureido]-3-fluorophenoxy}-pyridine-2-carboxylic acid methylamide and lopinavir and/or ritonavir is used.

The combination can comprise effective amounts of at least one compound of Formula I and at least one other therapeutic agent mentioned above, which achieves a greater therapeutic efficacy than when either compound is used alone. The combination can be useful to treat HIV infections and/or diseases caused by HIV infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating HIV infections and/or diseases caused by HIV infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and indinavir, zidovudine, tenofovir, parapoxvirus ovis and/or lamivudin. More preferably a combination comprising 4{4-[3-(4-chloro-3-trifluoromethylphenyl)-ureido]-3-fluorophenoxy}-pyridine-2-carboxylic acid methylamide and indinavir, zidovudine, tenofovir, parapoxvirus ovis and/or lamivudin is used.

The combination can comprise effective amounts of at least one compound of Formula I and at least one other therapeutic agent mentioned above, which achieves a greater therapeutic efficacy than when either compound is used alone. The combination can be useful to treat hepatitis virus infections and/or diseases caused by hepatitis virus infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating hepatitis virus infections and/or diseases caused by hepatitis virus infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and lamivudin and/or adevovir dipivoxil. More preferably a combination comprising 4{4-[3-(4-chloro-3-trifluoromethylphenyl)-ureido]-3-fluorophenoxy}-pyridine-2-carboxylic acid methylamide and lamivudin and/or adevovir dipivoxil is used.

The combination can comprise effective amounts of at least one compound of Formula I and at least one other therapeutic agent mentioned above, which achieves a greater therapeutic efficacy than when either compound is used alone. The combination can be useful to treat influenza virus infections and/or diseases caused by influenza virus infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating influenza virus infections and/or diseases caused by influenza virus infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and oseltamvir, zanamivir and/or pegylated interferon-α. More preferably a combination comprising 4{4-[3-(4-chloro-3-trifluoromethylphenyl)-ureido]-3-fluorophenoxy}-pyridine-2-carboxylic acid methylamide and oseltamvir, zanamivir and/or pegylated interferon-α is used.

The combination can comprise effective amounts of at least one compound of Formula I and at least one other therapeutic agent mentioned above, which achieves a greater therapeutic efficacy than when either compound is used alone. The combination can be useful to treat Herpesviridae viruses infections and/or diseases caused by Herpesviridae viruses infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating Herpesviridae viruses infections and/or diseases caused by Herpesviridae viruses infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and acyclovir. More preferably a combination comprising 4{4-[3-(4-chloro-3-trifluoromethylphenyl)-ureido]-3-fluorophenoxy}-pyridine-2-carboxylic acid methylamide and acyclovir.

The combination can comprise effective amounts of at least one compound of Formula I and at least one other therapeutic agent mentioned above, which achieves a greater therapeutic efficacy than when either compound is used alone. The combination can be useful to treat Papovaviridae viruses infections and/or diseases caused by Papovaviridae viruses infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating Papovaviridae viruses infections and/or diseases caused by Papovaviridae viruses infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and interferon, imiquimod, resiquimod, podophyllin, bleomycin and/or retinoid. More preferably a combination comprising 4{4-[3-(4-chloro-3-trifluoromethylphenyl)-ureido]-3-fluorophenoxy}-pyridine-2-carboxylic acid methylamide and interferon, imiquimod, resiquimod, podophyllin, bleomycin and/or retinoid.

The combination can comprise effective amounts of at least one compound of Formula I and at least one other therapeutic agent mentioned above, which achieves a greater therapeutic efficacy than when either compound is used alone. The combination can be useful to treat Poxyiridae viruses infections and/or diseases caused by Poxyiridae viruses infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating Poxyiridae viruses infections and/or diseases caused by Poxyiridae viruses infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and cidofovir, interferon-β, interferon alfacon-1, interferon-α and/or pegylated interferon-α. More preferably a combination comprising 4{4-[3-(4-chloro-3-trifluoromethylphenyl)-ureido]-3-fluorophenoxy}-pyridine-2-carboxylic acid methylamide and cidofovir, interferon-β, interferon alfacon-1, interferon-α and/or pegylated interferon-α is used.

The combination can comprise effective amounts of at least one compound of Formula I and at least one other therapeutic agent mentioned above, which achieves a greater therapeutic efficacy than when either compound is used alone. The combination can be useful to treat Flaviviridae viruses infections and/or diseases caused by Flaviviridae viruses infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating Flaviviridae viruses infections and/or diseases caused by Flaviviridae viruses infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and ribavirin, interferon-β, interferon alfacon-1, interferon-α and/or pegylated interferon-α. More preferably a combination comprising 4{4-[3-(4-chloro-3-trifluoromethylphenyl)-ureido]-3-fluorophenoxy}-pyridine-2-carboxylic acid methylamide and ribavirin, interferon-β, interferon alfacon-1, interferon-α and/or pegylated interferon-α is used.

The combination can comprise effective amounts of at least one compound of Formula I and at least one other therapeutic agent mentioned above, which achieves a greater therapeutic efficacy than when either compound is used alone. The combination can be useful to treat Virus infections according to the invention and/or diseases caused by such virus infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating virus infections according to the invention and/or diseases caused by such virus infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and interferon-β, interferon alfacon-1, interferon-α and/or pegylated interferon-α. More preferably a combination comprising 4{4-[3-(4-chloro-3-trifluoromethylphenyl)-ureido]-3-fluorophenoxy}-pyridine-2-carboxylic acid methylamide and interferon-β, interferon alfacon-1, interferon-α and/or pegylated interferon-α is used.

The combination can comprise effective amounts of at least one compound of Formula I and at least one other therapeutic agent mentioned above, which achieves a greater therapeutic efficacy than when either compound is used alone. The combination can be useful to treat Picornaviridae viruses infections and/or diseases caused by Picornaviridae viruses infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating Picornaviridae viruses infections and/or diseases caused by Picornaviridae viruses infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and ruprintrivir, pirodavir, parapoxvirus ovis and/or pegylated interferon-α. More preferably a combination comprising 4{4-[3-(4-chloro-3-trifluoromethylphenyl)-ureido]-phenoxy}-pyridine-2-carboxylic acid methyl amide (BAY 43-9006) or the p-toluenesulfonic acid salt of 4{4-[3-(4-chloro-3-trifluoromethylphenyl)-ureido]-phenoxy}-pyridine-2-carboxylic acid methyl amide and ruprintrivir, pirodavir, parapoxvirus ovis and/or pegylated interferon-α is used.

TABLE 1
(from the ICTVdB Index of Viruses on the worldwide web at
ncbi.nlm.nih.gov/ICTVdb/Ictv/fs_herpe.htm)

| | | |
|---|---|---|
| Family | 00.031. | Herpesviridae |
| Subfamily | 00.031.1. | Alphaherpesvirinae |
| Genus | 00.031.1.01. | Simplexvirus |
| Genus | 00.031.1.02. | Varicellovirus |
| Genus | 00.031.1.03. | Mardivirus (was “Marek's disease-like viruses”) |
| Genus | 00.031.1.04. | Iltovirus (was “Infectious laryngotracheitis-like viruses”) |
| Subfamily | 00.031.2. | Betaherpesvirinae |
| Genus | 00.031.2.01. | Cytomegalovirus |
| Genus | 00.031.2.02. | Muromegalovirus |
| Genus | 00.031.2.03. | Roseolovirus |
| Subfamily | 00.031.3. | Gammaherpesvirinae |
| Genus | 00.031.3.01. | Lymphocryptovirus |
| Genus | 00.031.3.02. | Rhadinovirus |
| Genus | 00.031.0.01. | Ictalurivirus (was “Ictalurid herpes-like viruses”) |
| Subfamily | 00.031.1. | Alphaherpesvirinae |
| Genus | 00.031.1.01. | Simplexvirus |
| Type Species | 00.031.1.01.001. | Human herpesvirus 1 (HHV-1) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, | 00.031.1.01.006. | Ateline herpesvirus 1 (AtHV-1) |
| their serotypes, | 00.031.1.01.006. | (Spider monkey herpesvirus) |
| strains and | 00.031.1.01.002. | Bovine herpesvirus 2 (BoHV-2) |
| isolates | 00.031.1.01.002. | (Bovine mamillitis virus) |
| | 00.031.1.01.005. | Cercopithecine herpesvirus 1 (CeHV-1) |
| | 00.031.1.01.005. | (B-virus) |
| | 00.031.1.01.005. | (Herpesvirus simiae) |
| | 00.031.1.01.007. | Cercopithecine herpesvirus 2 (CeHV-2) |
| | 00.031.1.01.007. | (SA8) |
| | 00.031.1.01.009. | Cercopithecine herpesvirus 16 (CeHV-16) |
| | 00.031.1.01.009. | (Herpesvirus papio 2) |
| | 00.031.1.01.003. | Human herpesvirus 1 [X14112] (HHV-1) |
| | 00.031.1.01.003. | (Herpes simplex virus 1) |
| | 00.031.1.01.004. | Human herpesvirus 2 [Z86099] (HHV-2) |
| | 00.031.1.01.004. | (Herpes simplex virus 2) |
| | 00.031.1.01.017. | Macropodid herpesvirus 1 (MaHV-1) |
| | 00.031.1.01.017. | (Parma wallaby herpesvirus) |
| | 00.031.1.01.018. | Macropodid herpesvirus 2 (MaHV-2) |
| | 00.031.1.01.018. | (Dorcopsis wallaby herpesvirus) |
| | 00.031.1.01.008. | Saimiriine herpesvirus 1 (SaHV-1) |
| | 00.031.1.01.008. | (Herpesvirus tamarinus) |
| | 00.031.1.01.008. | (Marmoset herpesvirus) |
| Genus | 00.031.1.02 | Varicellovirus |
| Type Species | 00.031.1.02.001. | Human herpesvirus 3 (HHV-3) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, | 00.031.1.02.002. | Bovine herpesvirus 1 [AJ004801] (BoHV-1) |
| their serotypes, | 00.031.1.02.002. | (Infectious bovine rhinotracheitis virus) |
| strains and | 00.031.1.02.003. | Bovine herpesvirus 5 (BoHV-5) |
| isolates | 00.031.1.02.003. | (Bovine encephalitis virus) |
| | 00.031.1.02.004. | Bubaline herpesvirus 1 (BuHV-1) |
| | 00.031.1.02.004. | (Water buffalo herpesvirus) |
| | 00.031.1.02.005. | Canid herpesvirus 1 (CaHV-1) |
| | 00.031.1.02.005. | (Canine herpesvirus) |
| | 00.031.1.02.006. | Caprine herpesvirus 1 (CpHV-1) |
| | 00.031.1.02.006. | (Goat herpesvirus) |
| | 00.031.1.02.007. | Cercopithecine herpesvirus 9 (CeHV-9) |
| | 00.031.1.02.007. | (Simian varicella virus) |
| | 00.031.1.02.007. | (Liverpool vervet herpesvirus) |
| | 00.031.1.02.007. | (Patas monkey herpesvirus delta) |
| | 00.031.1.02.007. | (Medical Lake macaque herpesvirus) |
| | 00.031.1.02.008. | Cervid herpesvirus 1 (CvHV-1) |
| | 00.031.1.02.008. | (Red deer herpesvirus) |
| | 00.031.1.02.009. | Cervid herpesvirus 2 (CvHV-2) |
| | 00.031.1.02.009. | (Reindeer herpesvirus) |
| | 00.031.1.02.010. | Equid herpesvirus 1 [M86664] (EHV-1) |
| | 00.031.1.02.010. | (Equine abortion virus) |
| | 00.031.1.02.018. | Equid herpesvirus 3 (EHV-3) |
| | 00.031.1.02.018. | (Equine coital exanthema virus) |
| | 00.031.1.02.011. | Equid herpesvirus 4 [AF030027] (EHV-4) |
| | 00.031.1.02.011. | (Equine rhinopneumonitis virus) |
| | 00.031.1.02.012. | Equid herpesvirus 8 (EHV-8) |
| | 00.031.1.02.012. | (Asinine herpesvirus 3) |
| | 00.031.1.02.013. | Equid herpesvirus 9 (EHV-9) |
| | 00.031.1.02.013. | (Gazelle herpesvirus) |
| | 00.031.1.02.014. | Felid herpesvirus 1 (FeHV-1) |
| | 00.031.1.02.014. | (Feline viral rhinotracheitis virus) |
| | 00.031.1.02.015. | Human herpesvirus 3 [X04370] (HHV-3) |
| | 00.031.1.02.015. | (Varicella-zoster virus) |
| | 00.031.1.02.016. | Phocid herpesvirus 1 (PhoHV-1) |
| | 00.031.1.02.016. | (Harbor seal herpesvirus) |
| | 00.031.1.02.017. | Suid herpesvirus 1 (SuHV-1) |
| | 00.031.1.02.017. | (Pseudorabies virus) (PRV) |
| Tentative Species | 00.031.1.82.019. | Equid herpesvirus 6 (EHV-6) |
| in the Genus | 00.031.1.82.019. | (Asinine herpesvirus 1) |
| Genus | 00.031.1.03. | Mardivirus (was “Marek's disease-like viruses)” |
| Type Species | 00.031.1.03.001. | Gallid herpesvirus 2 (GaHV-2) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, | 00.031.1.03.001. | Gallid herpesvirus 2 (GaHV-2) |
| their serotypes, | 00.031.1.03.001. | (Marek's disease herpesvirus 1) |
| strains and | 00.031.1.03.002. | Gallid herpesvirus 3 (GaHV-3) |
| isolates | 00.031.1.03.002. | (Marek's disease herpesvirus 2) |
| | 00.031.1.03.003. | Meleagrid herpesvirus 1 (MeHV-1) |
| | 00.031.1.03.003. | (Turkey herpesvirus 1) |
| Tentative Species | | |
| in the Genus | | |
| None reported. | | |
| Genus | 00.031.1.04. | Iltovirus (was “Infectious laryngotracheitis-like viruses”) |
| Type Species | 00.031.1.04.001. | Gallid herpesvirus 1 (GaHV-1) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, | 00.031.1.04.001. | Gallid herpesvirus 1 (GaHV-1) |
| their serotypes, | 00.031.1.04.001. | (Infectious laryngotracheitis virus) |
| strains and | | |
| isolates | | |
| Tentative Species | | |
| in the Genus | | |
| None reported. | | |
| List of Unassigned | 00.031.1.00.041. | Psittacid herpesvirus 1 (PsHV-1) |
| Viruses in | 00.031.1.00.041. | (Parrot herpesvirus) |
| the Subfamily | 00.031.1.00.041. | (Pacheco's disease virus) |
| Subfamily | 00.031.2. | Betaherpesvirinae |
| Genus | 00.031.2.01 | Cytomegalovirus |
| Type Species | 00.031.2.01.001. | Human herpesvirus 5 (HHV-5) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, | 00.031.2.01.002. | Cercopithecine herpesvirus 5 (CeHV-5) |
| their serotypes, | 00.031.2.01.002. | (African green monkey cytomegalovirus) |
| strains and | 00.031.2.01.003. | Cercopithecine herpesvirus 8 (CeHV-8) |
| isolates | 00.031.2.01.003. | (Rhesus monkey cytomegalovirus) |
| | 00.031.2.01.004. | Human herpesvirus 5 [X17403] (HHV-5) |
| | 00.031.2.01.004. | (Human cytomegalovirus) |
| | 00.031.2.01.005. | Pongine herpesvirus 4 (PoHV-4) |
| Tentative Species | 00.031.2.81.001. | Aotine herpesvirus 1 (AoHV-1) |
| in the Genus | 00.031.2.81.001. | (Herpesvirus aotus 1) |
| | 00.031.2.81.002. | Aotine herpesvirus 3 (AoHV-3) |
| | 00.031.2.81.002. | (Herpesvirus aotus 3) |
| Genus | 00.031.2.02. | Muromegalovirus |
| Type Species | 00.031.2.02.001. | Murid cytomegalovirus 1 (MCMV-1) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, | 00.031.2.02.001. | Murid herpesvirus 1 [U68299] (MuHV-1) |
| their serotypes, | 00.031.2.02.001. | (Mouse cytomegalovirus 1) |
| strains and | 00.031.2.02.002. | Murid herpesvirus 2 (MuHV-2) |
| isolates | 00.031.2.02.002. | (Rat cytomegalovirus) |
| Tentative Species | | |
| in the Genus | | |
| None reported | | |
| Genus | 00.031.2.03. | Roseolovirus |
| Type Species | 00.031.2.03.001. | Human herpesvirus 6 (HHV-6) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, | 00.031.2.03.001. | Human herpesvirus 6 (HHV-6) |
| their serotypes, | 00.031.2.03.001.00.001. | Human herpesvirus 6A (HHV-6A) |
| strains and | 00.031.2.03.001.00.001.001. | U1102 [X83413] |
| isolates | 00.031.2.03.001.00.002. | Human herpesvirus 6B (HHV-6B) |
| | 00.031.2.03.002. | Human herpesvirus 7 [U43400] (HHV-7) |
| | 00.031.2.03.002. | Human herpesvirus 7 [AF037218] |
| Tentative Species | | |
| in the Genus | | |
| None reported. | | |
| Subfamily | 00.031.3. | Gammaherpesvirinae |
| Genus | 00.031.3.01. | Lymphocryptovirus |
| Type Species | 00.031.3.01.001. | Human herpesvirus 4 (HHV-4) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, | 00.031.3.01.002. | Cercopithecine herpesvirus 12 (CeHV-12) |
| their serotypes, | 00.031.3.01.002. | (Herpesvirus papio) |
| strains and | 00.031.3.01.002. | (Baboon herpesvirus) |
| isolates | 00.031.3.01.003. | Cercopithecine herpesvirus 14 (CeHV-14) |
| | 00.031.3.01.003. | (African green monkey EBV-like virus) |
| | 00.031.3.01.004. | Cercopithecine herpesvirus 15 (CeHV-15) |
| | 00.031.3.01.004. | (Rhesus EBV-like virus) |
| | 00.031.3.01.005. | Human herpesvirus 4 [V01555] (HHV-4) |
| | 00.031.3.01.005. | (Epstein-Barr virus) |
| | 00.031.3.01.006. | Pongine herpesvirus 1 (PoHV-1) |
| | 00.031.3.01.006. | (Herpesvirus pan) |
| | 00.031.3.01.007. | Pongine herpesvirus 2 (PoHV-2) |
| | 00.031.3.01.007. | (Orangutan herpesvirus) |
| | 00.031.3.01.008. | Pongine herpesvirus 3 (PoHV-3) |
| | 00.031.3.01.008. | (Gorilla herpesvirus) |
| Tentative Species | | |
| in the Genus | | |
| None reported. | | |
| Genus | 00.031.3.02. | Rhadinovirus |
| Type Species | 00.031.3.02.001. | Saimiriine herpesvirus 2 (SaHV-2) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, | 00.031.3.02.003. | Alcelaphine herpesvirus 1 (AIHV-1) |
| their serotypes, | 00.031.3.02.003. | (Malignant catarrhal fever virus) |
| strains and | 00.031.3.02.004. | Alcelaphine herpesvirus 2 (AIHV-2) |
| isolates | 00.031.3.02.004. | (Hartebeest malignant catarrhal fever virus) |
| | 00.031.3.02.002. | Ateline herpesvirus 2 (AtHV-2) |
| | 00.031.3.02.002. | (Herpesvirus ateles) (AtHV-2) |
| | 00.031.3.02.005. | Bovine herpesvirus 4 (BoHV-4) |
| | 00.031.3.02.005. | (Movar virus) |
| | 00.031.3.02.006. | Cercopithecine herpesvirus 17 (CeHV-17) |
| | 00.031.3.02.006. | (Rhesus rhadinovirus) (CeHV-17) |
| | 00.031.3.02.007. | Equid herpesvirus 2 [U20824] (EHV-2) |
| | 00.031.3.02.008. | Equid herpesvirus 5 (EHV-5) |
| | 00.031.3.02.009. | Equid herpesvirus 7 (EHV-7) |
| | 00.031.3.02.009. | (Asinine herpesvirus 2) |
| | 00.031.3.02.010. | Hippotragine herpesvirus 1 (HiHV-1) |
| | 00.031.3.02.010. | (Roan antelope herpesvirus) |
| | 00.031.3.02.011. | Human herpesvirus 8 [U75699] (HHV-8) |
| | 00.031.3.02.011. | Human herpesvirus 8 [U75700] (HHV-8) |
| | 00.031.3.02.011. | Human herpesvirus 8 [U93872] (HHV-8) |
| | 00.031.3.02.011. | (Kaposi's sarcoma-associated herpesvirus) |
| | 00.031.3.02.012. | Murid herpesvirus 4 [U97553] (MuHV-4) |
| | 00.031.3.02.012. | (Mouse herpesvirus strain 68) |
| | 00.031.3.02.013. | Ovine herpesvirus 2 (OvHV-2) |
| | 00.031.3.02.013. | (Sheep-associated malignant catarrhal fever of cattle virus) |
| | 00.031.3.02.014. | Saimiriine herpesvirus 2 [X64346] (SaHV-2) |
| | 00.031.3.02.014. | (Herpesvirus saimiri) |
| Tentative Species | 00.031.3.82.015. | Leporid herpesvirus 1 (LeHV-1) |
| in the Genus | 00.031.3.82.015. | (Cottontail rabbit herpesvirus) |
| | 00.031.3.82.016. | Leporid herpesvirus 2 (LeHV-2) |
| | 00.031.3.82.016. | (Herpesvirus cuniculi) |
| | 00.031.3.82.017. | Leporid herpesvirus 3 (LeHV-1) |
| | 00.031.3.82.017. | (Herpesvirus sylvilagus) |
| | 00.031.3.82.018. | Marmomid herpesvirus 1 (MaHV-1) |
| | 00.031.3.82.018. | (Woodchuck herpesvirus marmota) |
| | 00.031.3.82.018. | (Herpesvirus marmota) |
| | 00.031.3.82.019. | Retroperitoneal fibromatosis-associated herpesvirus (RFHV) |
| List of | 00.031.3.00.006. | Callitrichine herpesvirus 1 (CalHV-1) |
| Unassigned Species | 00.031.3.00.006. | (Herpesvirus sanguinus) |
| in the Subfamily | 00.031.3.00.019. | Callitrichine herpesvirus 3 (CalHV-3) |
| | 00.031.3.00.020. | Mustelid herpesvirus 1 (MusHV-1) |
| Genus | 00.031.0.01 | Ictalurivirus (was “Ictalurid Herpes-like viruses”) |
| Type Species | 00.031.0.01.001. | Ictalurid herpesvirus 1 (IcHV-1) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, | 00.031.0.01.001. | Ictalurid herpesvirus 1 [M75136] (IcHV-1) |
| their serotypes, | 00.031.0.01.001. | (Channel catfish herpesvirus) (CCHV) |
| strains and | | |
| isolates | | |
| Tentative Species | | |
| in the Genus | | |
| None reported. | | |
| List of | 00.031.0.00.050. | Acipenserid herpesvirus 1 (AciHV-1) |
| Unassigned Viruses | 00.031.0.00.050. | (White sturgeon herpesvirus 1) |
| in the Family | 00.031.0.00.051. | Acipenserid herpesvirus 2 (AciHV-2) |
| | 00.031.0.00.051. | (White sturgeon herpesvirus 2) |
| | 00.031.0.00.001. | Acciptrid herpesvirus 1 (AcHV-1) |
| | 00.031.0.00.001. | (Bald eagle herpesvirus) |
| | 00.031.0.00.002. | Anatid herpesvirus 1 (AnHV-1) |
| | 00.031.0.00.002. | (Duck plague herpesvirus) |
| | 00.031.0.00.052. | Anguillid herpesvirus 1 (AngHV-1) |
| | 00.031.0.00.052. | (Japanese eel herpesvirus) |
| | 00.031.0.00.004. | Ateline herpesvirus 3 (AtHV-3) |
| | 00.031.0.00.004. | (Herpesvirus ateles strain 73) |
| | 00.031.0.00.005. | Boid herpesvirus 1 (BaHV-1) |
| | 00.031.0.00.005. | (Boa herpesvirus) |
| | 00.031.0.00.053. | Callitrichine herpesvirus 2 (CaHV-2) |
| | 00.031.0.00.053. | (Marmoset cytomegalovirus) |
| | 00.031.0.00.054. | Caviid herpesvirus 1 (CvHV-1) |
| | 00.031.0.00.054. | (Guinea pig herpesvirus) |
| | 00.031.0.00.054. | (Hsiung kaplow herpesvirus) |
| | 00.031.0.00.007. | Caviid herpesvirus 3 (CvHV-3) |
| | 00.031.0.00.007. | (Guinea pig herpesvirus 3) |
| | 00.031.0.00.055. | Cebine herpesvirus 1 (CbHV-1) |
| | 00.031.0.00.055. | (Capuchin herpesvirus AL-5) |
| | 00.031.0.00.056. | Cebine herpesvirus 2 (CbHV-2) |
| | 00.031.0.00.056. | (Capuchin herpesvirus AP-18) |
| | 00.031.0.00.057. | Cercopithecine herpesvirus 3 (CeHV-3) |
| | 00.031.0.00.057. | (SA6 virus) |
| | 00.031.0.00.058. | Cercopithecine herpesvirus 4 (CeHV-4) |
| | 00.031.0.00.058. | (SA 15 virus) |
| | 00.031.0.00.008. | Cercopithecine herpesvirus 10 (CeHV-10) |
| | 00.031.0.00.008. | (Rhesus leukocyte associated herpesvirus strain 1) |
| | 00.031.0.00.009. | Cercopithecine herpesvirus 13 (CeHV-13) |
| | 00.031.0.00.009. | (Herpesvirus cyclopsis) |
| | 00.031.0.00.011. | Chelonid herpesvirus 1 (ChHV-1) |
| | 00.031.0.00.011. | (Gray patch disease of turtles) |
| | 00.031.0.00.012. | Chelonid herpesvirus 2 (ChHV-2) |
| | 00.031.0.00.012. | (Pacific pond turtle herpesvirus) |
| | 00.031.0.00.013. | Chelonid herpesvirus 3 (ChHV-3) |
| | 00.031.0.00.013. | (Painted turtle herpesvirus) |
| | 00.031.0.00.014. | Chelonid herpesvirus 4 (ChHV-4) |
| | 00.031.0.00.014. | (Argentine turtle herpesvirus) |
| | 00.031.0.00.015. | Ciconiid herpesvirus 1 (CiHV-1) |
| | 00.031.0.00.015. | (Black stork herpesvirus) |
| | 00.031.0.00.016. | Columbid herpesvirus 1 (CoHV-1) |
| | 00.031.0.00.016. | (Pigeon herpesvirus) |
| | 00.031.0.00.059. | Cricetid herpesvirus (CrHV-1) |
| | 00.031.0.00.059. | (Hamster herpesvirus) |
| | 00.031.0.00.017. | Cyprinid herpesvirus 1 (CyHV-1) |
| | 00.031.0.00.017. | (Carp pox herpesvirus) |
| | 00.031.0.00.060. | Cyprinid herpesvirus 2 (CyHV-2) |
| | 00.031.0.00.060. | (Goldfish herpesvirus) |
| | 00.031.0.00.060. | (Haematopoietic necrosis herpesvirus of goldfish) |
| | 00.031.0.00.019. | Elapid herpesvirus 1 (EpHV-1) |
| | 00.031.0.00.019. | (Indian cobra herpesvirus) |
| | 00.031.0.00.019. | (Banded krait herpesvirus) |
| | 00.031.0.00.019. | (Siamese cobra herpesvirus) |
| | 00.031.0.00.018. | Elephantid herpesvirus 1 (EiHV-1) |
| | 00.031.0.00.018. | (Elephant loxondontal herpesvirus) |
| | 00.031.0.00.020. | Erinaceid herpesvirus 1 (ErHV-1) |
| | 00.031.0.00.020. | (European hedgehog herpesvirus) |
| | 00.031.0.00.021. | Esocid herpesvirus 1 (EsHV-1) |
| | 00.031.0.00.021. | (Northern pike herpesvirus) |
| | 00.031.0.00.022. | Falconid herpesvirus 1 (FaHV-1) |
| | 00.031.0.00.022. | (Falcon inclusion body disease) |
| | 00.031.0.00.025. | Gruid herpesvirus 1 (GrHV-1) |
| | 00.031.0.00.025. | (Crane herpesvirus) |
| | 00.031.0.00.029. | Lacertid herpesvirus 1 (LaHV-1) |
| | 00.031.0.00.029. | (Green lizard herpesvirus) |
| | 00.031.0.00.028. | Lorisine herpesvirus 1 (LoHV-1) |
| | 00.031.0.00.028. | (Kinkajou herpesvirus) |
| | 00.031.0.00.028. | (Herpesvirus pottos) |
| | 00.031.0.00.031. | Murid herpesvirus 3 (MuHV-3) |
| | 00.031.0.00.031. | (Mouse thymic herpesvirus) |
| | 00.031.0.00.032. | Murid herpesvirus 5 (MuHV-5) |
| | 00.031.0.00.032. | (Field mouse herpesvirus) |
| | 00.031.0.00.032. | (Microtus pennsylvanicus herpesvirus) |
| | 00.031.0.00.033. | Murid herpesvirus 6 (MuHV-6) |
| | 00.031.0.00.033. | (Sand rat nuclear inclusion agents) |
| | 00.031.0.00.061. | Ostreid herpesvirus 1 (OsHV-1) |
| | 00.031.0.00.061. | (Pacific oyster herpesvirus) |
| | 00.031.0.00.035. | Ovine herpesvirus 1 (OvHV-1) |
| | 00.031.0.00.035. | (Sheep pulmonary adenomatosis associated herpesvirus) |
| | 00.031.0.00.036. | Percid herpesvirus 1 (PeHV-1) |
| | 00.031.0.00.036. | (Walleye epidermal hyperplasia) |
| | 00.031.0.00.037. | Perdicid herpesvirus 1 (PdHV-1) |
| | 00.031.0.00.037. | (Bobwhite quail herpesvirus) |
| | 00.031.0.00.038. | Phalacrocoracid herpesvirus 1 (PhHV-1) |
| | 00.031.0.00.038. | (Cormorant herpesvirus) |
| | 00.031.0.00.038. | (Lake Victoria cormorant herpesvirus) |
| | 00.031.0.00.040. | Pleuronectid herpesvirus (PiHV-1) |
| | 00.031.0.00.040. | (Herpesvirus scophthalmus) |
| | 00.031.0.00.040. | (Turbot herpesvirus) |
| | 00.031.0.00.042. | Ranid herpesvirus 1 (RaHV-1) |
| | 00.031.0.00.042. | (Lucke frog herpesvirus) |
| | 00.031.0.00.043. | Ranid herpesvirus 2 (RaHV-2) |
| | 00.031.0.00.043. | (Frog herpesvirus 4) |
| | 00.031.0.00.044. | Salmonid herpesvirus 1 (SaHV-1) |
| | 00.031.0.00.044. | (Herpesvirus salmonis) |
| | 00.031.0.00.045. | Salmonid herpesvirus 2 (SaHV-2) |
| | 00.031.0.00.045. | (Onchorhynchus masou herpesvirus) |
| | 00.031.0.00.063. | Sciurid herpesvirus 1 (ScHV-1) |
| | 00.031.0.00.063. | (European ground squirrel cytomegalovirus) |
| | 00.031.0.00.046. | Sciurid herpesvirus 2 (ScHV-2) |
| | 00.031.0.00.046. | (American ground squirrel herpesvirus) |
| | 00.031.0.00.047. | Sphenicid herpesvirus 1 (SpHV-1) |
| | 00.031.0.00.047. | (Black footed penguin herpesvirus) |
| | 00.031.0.00.048. | Strigid herpesvirus 1 (StHV-1) |
| | 00.031.0.00.048. | (Owl hepatosplenitis herpesvirus) |
| | 00.031.0.00.062. | Suid herpesvirus 2 (SuHV-2) |
| | 00.031.0.00.062. | (Swine cytomegalovirus) |
| | 00.031.0.00.049. | Tupaiid herpesvirus 1 (TuHV-1) |
| | 00.031.0.00.049. | (Tree shrew herpesvirus) |

TABLE 2
(from worldwide web at ncbi.nlm.nih.gov/ICTVdb/Ictv/fs_papov.htm)

```
Family Papovaviridae
    1. Genus Polyomavirus
    2. Genus Papillomavirus
Genus Polyomavirus
Type Species
  murine polyomavirus (strain A2) (PyV)
Taxonomic Structure of the Genus
Species in the Genus
Virus name (synonym) followed by [Genomic sequence accession number] (Acronym)
    African green monkey polyomavirus (LPV)
      (B-lymphotropic papovavirus strain K38) [K02562] baboon polyomavirus 2 (PPV-2)
    BK virus (strain Dun) [J02038] (BKV)
    bovine polyomavirus (BPyV)
      (stump-tailed macaque virus)
      (fetal rhesus kidney virus) [D00755] budgerigar fledgling disease virus (BFDV)
    hamster polyomavirus [X02449] (HaPV)
    JC virus (strain Mad1) [J02226] (JCV)
    murine polyomavirus [M55904] (KV)
      (mice pneumotropic virus)
      (Kilham strain, or K virus) murine polyomavirus (strain A2) [J02288] (PyV)
    rabbit kidney vacuolating virus (RKV)
    simian agent virus 12 (SAV-12)
    simian virus 40 (strain 776) [J02400] (SV-40)
Genus Papillomavirus
Type Species
  cottontail rabbit papillomavirus (Shope) (CRPV)
Taxonomic Structure of the Genus
Members of this genus are known from humans (more than 63 types, HPV-1, etc.), chimpanzee,
colobus and rhesus monkeys, cow (6 types), deer, dog, horse, sheep, elephant, elk, opossum,
multimammate and European harvest mouse, turtle, chaffinch and parrot.
Species in the Genus
Virus name (synonym) followed by [Genomic sequence accession number] (Acronym)
    bovine papillomavirus 1 [X02346] (BPV-1)
    bovine papillomavirus 2 [M20219] (BPV-2)
    bovine papillomavirus 4 [X05817] (BPV-4)
    canine oral papillomavirus (COPV)
    chaffinch papillomavirus (ChPV)
    cottontail rabbit papillomavirus (Shope) [K02708] (CRPV)
    deer papillomavirus [M11910] (DPV)
      (deer fibroma virus) elephant papillomavirus (EPV)
    equine papillomavirus (EqPV)
    European elk papillomavirus [M15953] (EEPV)
    human papillomavirus 1a [V01116] (HPV-1a)
    human papillomavirus 5 (HPV-5)
    human papillomavirus 6b (HPV-6b)
    human papillomavirus 8 (HPV-8)
    human papillomavirus 11 [M14119] (HPV-11)
    human papillomavirus 16 [K02718] (HPV-16)
    human papillomavirus 18 [X05015] (HPV-18)
    human papillomavirus 31 [J04353] (HPV-31)
    human papillomavirus 33 [M12732] (HPV-33)
    multimammate mouse papillomavirus (MnPV)
    rabbit oral papillomavirus (ROPV)
    reindeer papillomavirus (RePV)
    rhesus monkey papillomavirus (RMPV)
    sheep papillomavirus (SPV)
```

TABLE 3
HPV type and disease association
(from Burd et al., Clinical Microbiol. Rev., 16: 1-17, Jan. 2003)
Order indicates relative frequency; bold and underline indicate most
frequent association

| Disease | HPV type |
|---|---|
| Plantar warts | 1, 2, 4, 63 |
| Common warts | 2, 1, 7, 4, 26, 27, 29, 41, 57, 65, 77, 1, 3, 4, |
| | 10, 28 |
| Flat warts | 3, 10, 26, 27, 28, 38, 41, 49, 75, 76 |
| Other cutaneous lesions | 6, 11, 16, 30, 33, 36, 37, 38, 41, 48, |
| (e.g., epidermoid cysts, | 60, 72, 73 |
| laryngeal carcinoma) | |
| Epidermodysplasia | 2, 3, 10, 5, 8, 9, 12, 14, 15, 17, 19, 20, 21, |
| verruciformis | 22, 23, 24, 25, 36, 37, 38, 47, 50 |
| Recurrent respiratory | 6, 11 |
| papillomatosis | |
| Focal epithelial hyperplasia | 13, 32 |
| of Heck | |
| Conjunctival | 6, 11, 16 |
| papillomas/carcinomas | |
| Condyloma acuminata | 6, 11, 30, 42, 43, 45, 51, 54, 55, 70 |
| (genital warts) | |
| Cervical intraepithelial | |
| neoplasia | |
| Unspecified | 30, 34, 39, 40, 53, 57, 59, 61, 62, 64, |
| | 66, 67, 68, 69 |
| Low risk | 6, 11, 16, 18, 31, 33, 35, 42, 43, 44, 45, 51, |
| | 52, 74 |
| High risk | 16, 18, 6, 11, 31, 34, 33, 35, 39, 42, 44, |
| | 45, 51, 52, 56, 58, 66 |
| Cervical carcinoma | 16, 18, 31, 45, 33, 35, 39, 51, 52, 56, 58, |
| | 66, 68, 70 |

TABLE 4
(from the ICTVdB Index of Viruses on the worldwide web at
ncbi.nlm.nih.gov/ICTVdb/Ictv/fs_poxvi.htm)
Taxonomic Structure of the Family

| | | |
|---|---|---|
| Family | 00.058. | Poxviridae |
| Subfamily | 00.058.1. | Chordopoxvirinae |
| Genus | 00.058.1.01. | Orthopoxvirus |
| Genus | 00.058.1.02. | Parapoxvirus |
| Genus | 00.058.1.03. | Avipoxvirus |
| Genus | 00.058.1.04. | Capripoxvirus |
| Genus | 00.058.1.05. | Leporipoxvirus |
| Genus | 00.058.1.06. | Suipoxvirus |
| Genus | 00.058.1.07. | Molluscipoxvirus |
| Genus | 00.058.1.08. | Yatapoxvirus |
| | 00.058.1.00. | Unassigned viruses in the Subfamily |
| Subfamily | 00.058.2. | Entomopoxvirinae |
| Genus | 00.058.2.01. | Alphaentomopoxvirus |
| Genus | 00.058.2.02. | Betaentomopoxvirus |
| Genus | 00.058.2.03. | Gammaentomopoxvirus |
| | 00.058.2.00. | Unassigned viruses in the Family |
| Subfamily | 00.058.1. | Chordopoxvirinae |
| Genus | 00.058.1.01. | Orthopoxvirus |
| Type Species | 00.058.1.01.001. | Vaccinia virus (VACV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:

| | | |
|---|---|---|
| Species, their serotypes, strains and isolates | 00.058.1.01.003. | Camelpox virus [S51129] (CMLV) |
| | 00.058.1.01.003. | {camel} |
| | 00.058.1.01.004. | Cowpox virus [M19531] (CPXV) |
| | 00.058.1.01.004. | {rodents, felines, bovines, human} |
| | 00.058.1.01.005. | Ectromelia virus [M83102] (ECTV) |
| | 00.058.1.01.005. | (Mousepox) |
| | 00.058.1.01.005. | {reservoir unknown} |
| | 00.058.1.01.006. | Monkeypox virus [K02025] (MPXV) |
| | 00.058.1.01.006. | {rodents, primates, human} |
| | 00.058.1.01.008. | Raccoonpox virus [M94169] (RCNV) |
| | 00.058.1.01.008. | {North America raccoon} |
| | 00.058.1.01.009. | Taterapox virus (GBLV) |
| | 00.058.1.01.009. | {African gerbil} |
| | 00.058.1.01.010. | Vaccinia virus [M35027] (VACV) |
| | 00.058.1.01.010. | {no natural reservoir} |
| | 00.058.1.01.010.01. | Buffalopox virus [U87233] (BPXV) |
| | 00.058.1.01.010.01. | {buffalo, cattle, human} |
| | 00.058.1.01.010.02. | Rabbitpox virus [M60387] (RPXV) |
| | 00.058.1.01.010.02. | {colonized rabbit, no natural reservoir} |
| | 00.058.1.01.011. | Variola virus [K02031] (VARV) |
| | 00.058.1.01.011. | {human; eradicated from nature} |
| | 00.058.1.01.012. | Volepox virus (VPXV) |
| | 00.058.1.01.012. | {California pinon mouse and voles} |
| Tentative Species in the Genus | 00.058.1.81.013. | Skunkpox virus (SKPV) |
| | 00.058.1.81.013. | {North American striped skunk} |
| | 00.058.1.81.014. | Uasin Gishu disease virus (UGDV) |
| | 00.058.1.81.014. | {Central African horses} |
| Genus | 00.058.1.02. | Parapoxvirus |
| Type Species | 00.058.1.02.001. | Orf virus (ORF) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:

| | | |
|---|---|---|
| Species, their serotypes, strains and isolates | 00.058.1.02.002. | Bovine papular stomatitis virus (BPSV) |
| | 00.058.1.02.002. | {bovines, human} |
| | 00.058.1.02.003. | Orf virus [M30023] (ORFV) |
| | 00.058.1.02.003. | (Contagious pustular dermatitis virus) |
| | 00.058.1.02.003. | (Contagious ecthyma virus) |
| | 00.058.1.02.003. | {Sheep, goats, musk oxen, human, deer} |
| | 00.058.1.02.004. | Parapoxvirus of red deer in New Zealand (PVNZ) |
| | 00.058.1.02.005. | Pseudocowpox virus (PCPV) |
| | 00.058.1.02.005. | (Milker's nodule virus) |
| | 00.058.1.02.005. | (ParaVaccinia virus) |
| | 00.058.1.02.005. | {Bovines, human} |
| | 00.058.1.02.006. | Squirrel parapoxvirus (SPPV) |
| Tentative Species in the Genus | 00.058.1.82.007. | Auzduk disease virus |
| | 00.058.1.82.007. | (Camel contagious ecthyma virus) |
| | 00.058.1.82.008. | Chamois contagious ecthyma virus |
| | 00.058.1.82.009. | Sealpox virus |
| Genus | 00.058.1.03. | Avipoxvirus |
| Type Species | 00.058.1.03.001. | Fowlpox virus (FWPV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:

| | | |
|---|---|---|
| Species, their serotypes, strains and isolates | 00.058.1.03.002. | Canarypox virus (CNPV) |
| | 00.058.1.03.003. | Fowlpox virus [X17202] (FWPV) |
| | 00.058.1.03.003. | Fowlpox virus [D00295] (FWPV) |
| | 00.058.1.03.003. | Fowlpox virus [AF198100] (FWPV) |
| | 00.058.1.03.004. | Juncopox virus (JNPV) |
| | 00.058.1.03.005. | Mynahpox virus (MYPV) |
| | 00.058.1.03.006. | Pigeonpox virus [M88588] (PGPV) |
| | 00.058.1.03.007. | Psittacinepox virus (PSPV) |
| | 00.058.1.03.008. | Quailpox virus (QUPV) |
| | 00.058.1.03.009. | Sparrowpox virus (SRPV) |
| | 00.058.1.03.010. | Starlingpox virus (SLPV) |
| | 00.058.1.03.011. | Turkeypox virus (TKPV |
| Tentative Species in the Genus | 00.058.1.83.012. | Crowpox virus (CRPV |
| | 00.058.1.83.013. | Peacockpox virus (PKPV |
| | 00.058.1.83.014. | Penguinpox virus (PEPV) |
| Genus | 00.058.1.04. | Capripoxvirus |
| Type Species | 00.058.1.04.001. | Sheeppox virus (SPPV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:

| | | |
|---|---|---|
| Species, their serotypes, strains and isolates | 00.058.1.04.002. | Goatpox virus (GTPV) |
| | 00.058.1.04.003. | Lumpy skin disease virus (LSDV) |
| | 00.058.1.04.004. | Sheeppox virus [M28823] (SPPV) |
| | 00.058.1.04.004. | Sheeppox virus [M30039] (SPPV) |
| | 00.058.1.04.004. | Sheeppox virus [D00423] (SPPV) |
| | 00.058.1.04.004. | Sheeppox virus [S78201] (SPPV) |
| Tentative Species in the Genus | | |
| None reported. | | |
| Genus | 00.058.1.05. | Leporipoxvirus |
| Type Species | 00.058.1.05.001. | Myxoma virus (MYXV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:

| | | |
|---|---|---|
| Species, their serotypes, strains and isolates | 00.058.1.05.002. | Hare fibroma virus (FIBV) |
| | 00.058.1.05.002. | {European hare} |
| | 00.058.1.05.003. | Myxoma virus [M93049] (MYXV) |
| | 00.058.1.05.004. | Rabbit fibroma virus [M14899] (SFV) |
| | 00.058.1.05.004. | (Shope fibroma virus) |
| | 00.058.1.05.005. | Squirrel fibroma virus (SQFV) |
| Tentative Species in the Genus | | |
| None reported. | | |
| Genus | 00.058.1.06. | Suipoxvirus |
| Type Species | 00.058.1.06.001. | Swinepox virus (SWPV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:

| | | |
|---|---|---|
| Species, their serotypes, strains and isolates | 00.058.1.06.001. | Swinepox virus [M59931] (SWPV) |
| | 00.058.1.06.001. | Swinepox virus [M64000] (SWPV) |
| Tentative Species in the Genus | | |
| None reported. | | |
| Genus | 00.058.1.07. | Molluscipoxvirus |
| Type Species | 00.058.1.07.001. | Molluscum contagiosum virus (MOCV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:

| | | |
|---|---|---|
| Species, their serotypes, strains and isolates | 00.058.1.07.001. | Molluscum contagiosum virus [M63487] (MOCV) |
| | 00.058.1.07.001. | Molluscum contagiosum virus [U60315] (MOCV) |
| Tentative Species in the Genus | | |
| Unnamed viruses | | |
| of horses, donkeys, | | |
| chimpanzees | | |
| Genus | 00.058.1.08. | Yatapoxvirus |
| Type Species | 00.058.1.08.001. | Yaba monkey tumor virus (YMTV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:

| | | |
|---|---|---|
| Species, their serotypes, strains and isolates | 00.058.1.08.002. | Tanapox virus (TANV) |
| | 00.058.1.08.003. | Yaba monkey tumor virus [D26580] (YMTV) |
| Tentative Species in the Genus | | |
| None reported. | | |

List of Unassigned Viruses in the Subfamily
The viruses, their host { } and assigned abbreviations ( ) are:

| | | |
|---|---|---|
| | 00.058.1.00.001. | California harbor seal poxvirus (SPV) |
| | | {May also infect dog, cat} |
| | 00.058.1.00.002. | Cotia virus [D45170] (CPV) |
| | | {sentinel mice, Brazil} |
| | 00.058.1.00.003. | Dolphin poxvirus (DOV) |
| | | {Bottle-nose dolphin} |
| | 00.058.1.00.004. | Embu virus (ERV) |
| | | {Mosquitoes, Human blood} |
| | 00.058.1.00.005. | Grey kangaroo poxvirus (KXV) |
| | 00.058.1.00.006. | Marmoset poxvirus (MPV) |
| | 00.058.1.00.007. | Molluscum-like poxvirus (MOV) |
| | | {Horse, donkey, chimpanzee} |
| | 00.058.1.00.014. | Mule deer poxvirus (DPV) |
| | | {Odocoileus hemionus, Wyoming} |
| | 00.058.1.00.008. | Nile crocodile poxvirus (CRV) |
| | 00.058.1.00.009. | Quokka poxvirus (QPV) |
| | | {marsupial, Australia} |
| | 00.058.1.00.010. | Red kangaroo poxvirus (KPV) |
| | 00.058.1.00.011. | Salanga poxvirus (SGV) |
| | | {Aethomys medicatus, Cent. Afr. Rep} |
| | 00.058.1.00.012. | Spectacled caiman poxvirus (RPV) |
| | 00.058.1.00.013. | Vole poxvirus (VPV) |
| | | {vole, Turkmenia} |
| | 00.058.1.00.015. | Yoka poxvirus (YKV) |
| | | {Aedes simpsoni, Centr. Afr. Rep.} |
| Subfamily | 00.058.2. | Entomopoxvirinae |
| Genus | 00.058.2.01. | Alphaentomopoxvirus |
| Type Species | 00.058.2.01.001. | Melolontha melolontha entomopoxvirus (MMEV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:

| | | |
|---|---|---|
| Species, their serotypes, strains and isolates | 00.058.2.01.002. | Anomala cuprea entomopoxvirus (ACEV) |
| | 00.058.2.01.003. | Aphodius tasmaniae entomopoxvirus (ATEV) |
| | 00.058.2.01.004. | Demodema boranensis entomopoxvirus (DBEV) |
| | 00.058.2.01.005. | Dermolepida albohirtum entomopoxvirus (DAEV) |
| | 00.058.2.01.006. | Figulus subleavis entomopoxvirus (FSEV) |
| | 00.058.2.01.007. | Geotrupes sylvaticus entomopoxvirus (GSEV) |
| | 00.058.2.01.008. | Melolontha melolontha entomopoxvirus [X77616] (MMEV) |
| | 00.058.2.01.009 | Othnonius batesi entomopoxvirus (ObEPV) |
| {O. batesi (coleoptera)} | 00.058.2.01.010 | Phyllopertha horticola entomopoxvirus (PhEPV) |
| | | {P. horticola (Coleoptera)} |
| Tentative Species in the Genus | | |
| ICTV reports none. | 00.058.2.81.011. | Ips typographus entomopoxvirus (ItEPV) |
| | | {I. typographus (coleoptera)} |
| Genus | 00.058.2.02. | Betaentomopoxvirus |
| Type Species | 00.058.2.02.001. | Amsacta moorei entomopoxvirus (AMEV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], their ‘origins L = lepidopteran,
O = orthopteran’ and assigned abbreviations ( ), are:

| | | |
|---|---|---|
| | 00.058.2.02.002. | Acrobasis zelleri entomopoxvirus ‘L’ (AZEV) |
| | 00.058.2.02.001. | Amsacta moorei entomopoxvirus ‘L’ [M80924] (AMEV) |
| | 00.058.2.02.001. | Amsacta moorei entomopoxvirus ‘L’ [M77182] (AMEV) |
| | 00.058.2.02.003. | Arphia conspersa entomopoxvirus ‘O’ (ACOEV) |
| | 00.058.2.02.004. | Choristoneura biennis entomopoxvirus ‘L’ [M34140] (CBEV) |
| | 00.058.2.02.004. | Choristoneura biennis entomopoxvirus ‘L’ [D10680] (CBEV) |
| | 00.058.2.02.005. | Choristoneura conflicta entomopoxvirus ‘L’ (CCEV) |
| | 00.058.2.02.006. | Choristoneura diversuma entomopoxvirus ‘L’ (CDEV) |
| | 00.058.2.02.013. | Choristoneura fumiferana entomopoxvirus ‘L’ [D10681] (CFEV) |
| | 00.058.2.02.013. | Choristoneura fumiferana entomopoxvirus ‘L’ [U10476] (CFEV) |
| | 00.058.2.02.007. | Chorizagrotis auxiliars entomopoxvirus ‘L’ (CXEV) |
| | 00.058.2.02.014. | Heliothis armigera entomopoxvirus ‘L’ [AF019224] (HAEV) |
| | 00.058.2.02.014. | Heliothis armigera entomopoxvirus ‘L’ [L08077] (HAEV) |
| | 00.058.2.02.008. | Locusta migratoria entomopoxvirus ‘O’ (LMEV) |
| | 00.058.2.02.010. | Oedaleus senigalensis entomopoxvirus ‘O’ (OSEV) |
| | 00.058.2.02.011. | Operopbtera brumata entomopoxvirus ‘L’ (OBEV) |
| | 00.058.2.02.012. | Schistocera gregaria entomopoxvirus ‘O’ (SGEV) |
| Tentative Species in the Genus | 00.058.2.82.013. | Pseudaletia separata entomopoxvirus ‘L’ (PsEPV) |
| | | {P. separata (Lepidoptera)} |
| Genus | 00.058.2.03. | Gammaentomopoxvirus |
| Type Species | 00.058.2.03.001. | Chironomus luridus entomopoxvirus CLEV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:

| | | |
|---|---|---|
| Species, their serotypes, strains and isolates | 00.058.2.03.002. | Aedes aegypti entomopoxvirus (AAEV) |
| | 00.058.2.03.003. | Camptochironomus tentans entomopoxvirus (CTEV) |
| | 00.058.2.03.004. | Chironomus attenuatus entomopoxvirus (CAEV) |
| | 00.058.2.03.005. | Chironomus luridus entomopoxvirus (CLEV) |
| | 00.058.2.03.006. | Chironomus plumosus entomopoxvirus (CPEV) |
| | 00.058.2.03.007. | Goeldichironomus haloprasimus entomopoxvirus (GHEV) |
| Tentative Species in the Genus | | |
| None reported. | | |

List of Unassigned Viruses in the Subfamily
The viruses, their host { } and assigned abbreviations ( ) are:

| | |
|---|---|
| 00.058.2.00.001. | Diachasmimorpha entomopoxvirus (DIEVV) |
| 00.058.2.00.009. | Melanoplus sanguinipes entomopoxvirus ‘O’ [AF063866] (MSEV) |

TABLE 5
(from the ICTVdB Index of Viruses on the worldwide web at
ncbi.nlm.nih.gov/ICTVdb/Ictv/fs_herpe.htm)
Taxonomic Structure of the Family

| | | |
|---|---|---|
| Family | 00.026. | Flaviviridae |
| Genus | 00.026.0.01. | Flavivirus |
| Genus | 00.026.0.02. | Pestivirus |
| Genus | 00.026.0.03. | Hepacivirus |
| Genus | 00.026.0.01. | Flavivirus |
| Type Species | 00.026.0.01.001. | Yellow fever virus (YFV) |

List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:
Species, their serotypes, strains and isolates
1. Tick-borne viruses

| | | |
|---|---|---|
| Mammalian tick-borne virus group | 00.026.0.01.016. | Gadgets Gully virus [AF013374] (GGYV) |
| | 00.026.0.01.022. | Kadam virus [AF013380] (KADV) |
| | 00.026.0.01.026. | Kyasanur Forest disease virus [X74111] (KFDV) |
| | 00.026.0.01.027. | Langat virus (LGTV) |
| | 00.026.0.01.027.02.102.002. | strain TP21 [M73835] |
| | 00.026.0.01.027.02.102.002. | strain TP21 [M86650] |
| | 00.026.0.01.034. | Omsk hemorrhagic fever virus [X66694] (OHFV) |
| | 00.026.0.01.036. | Powassan virus [L06436] (POWV) |
| | 00.026.0.01.038. | Royal Farm virus [AF013398] (RFV) |
| | 00.026.0.01.038.02.102.003. | Karshi virus [AF013381] (KSIV) |
| | 00.026.0.01.046. | Tick-borne encephalitis virus (TBEV) |
| | 00.026.0.01.046.02.101. | European subtype |
| | 00.026.0.01.046.02.101.004. | Neudoerfl virus [M27157] (NEUV) |
| | 00.026.0.01.046.02.101.004. | Neudoerfl virus [M33668] (NEUV) |
| | 00.026.0.01.046.02.102. | Far Eastern subtype [X07755] |
| | 00.026.0.01.046.02.102.003. | Sofjin virus [X07755] (SOFV) |
| | 00.026.0.01.046.02.103. | Sibirian subtype |
| | 00.026.0.01.046.02.103.001. | Vasilchenko [L40361] |
| | 00.026.0.01.028. | Louping ill virus (LIV) |
| | 00.026.0.01.028.02.100.002. | LIV strain 369/T2 [M59376] |
| | 00.026.0.01.028.02.100.003. | LIV strain SB 526 [M94957] |
| | 00.026.0.01.028.02.100.003. | LIV strain SB 526 [X59815] |
| | 00.026.0.01.028.02.100.007. | Negishi virus [M94956] (NEGV) |
| | 00.026.0.01.028.02.101. | Irish subtype [X86784] |
| | 00.026.0.01.028.02.102. | British subtype [D12937] |
| | 00.026.0.01.028.02.103. | Spanish subtype [X77470] |
| | 00.026.0.01.028.02.104. | Turkish subtype [X69125] |
| Seabird tick-borne virus group | 00.026.0.01.029. | Meaban virus (MEAV) |
| | 00.026.0.01.029.05.105.001. | Brest ART707 [AF013386] |
| | 00.026.0.01.042. | Saumarez Reef virus (SREV) |
| | 00.026.0.01.042.05.105.001. | CSIRO-4 [X80589] |
| | 00.026.0.01.047. | Tyuleniy virus (TYUV) |
| | 00.026.0.01.047.05.105.001. | Three Arch Rock [X80588] |

2. Mosquito-borne viruses

| | | |
|---|---|---|
| Aroa virus group | 00.026.0.01.003. | Aroa virus (AROAV) |
| | 00.026.0.01.003.03.001.001. | VenA-1809 [AF013362] |
| | 00.026.0.01.003.03.002. | Bussuquara virus (BSQV) |
| | 00.026.0.01.003.03.002.001. | BeAn 4073 [AF013366] |
| | 00.026.0.01.003.03.003. | Iguape virus (IGUV) |
| | 00.026.0.01.003.03.003.001. | SP An71686 [AF013375] |
| | 00.026.0.01.003.03.004. | Naranjal virus (NJLV) |
| | 00.026.0.01.003.03.004.001. | 25008 [AF013390] |
| Dengue virus group | 00.026.0.01.013. | Dengue virus (DENV) |
| | 00.026.0.01.013.08.201. | Dengue virus 1 [M23027] (DENV-1) |
| | 00.026.0.01.013.08.202. | Dengue virus 2 [M19197] (DENV-2) |
| | 00.026.0.01.013.08.203. | Dengue virus 3 [A34774] (DENV-3) |
| | 00.026.0.01.013.08.204. | Dengue virus 4 [M14931] (DENV-4) |
| | 00.026.0.01.023. | Kedougou virus (KEDV) |
| | 00.026.0.01.023.08.202.001. | Dak Aar D1470 [AF013382] |
| Japanese encephalitis virus group | 00.026.0.01.009. | Cacipacore virus (CPCV) |
| | 00.026.0.01.009.03.000.001. | BeAn 327600 [AF013367] |
| | 00.026.0.01.025. | Koutango virus (KOUV) |
| | 00.026.0.01.025.04.204.001. | Dak Ar D1470 [AF013384] |
| | 00.026.0.01.019. | Japanese encephalitis virus (JEV) |
| | 00.026.0.01.019.04.204.001. | strain JaOArS982 [M18370] |
| | 00.026.0.01.032. | Murray Valley encephalitis virus [X03467] (MVEV) |
| | 00.026.0.01.032.04.204.002. | Alfuy virus [AF013360] (ALFV) |
| | 00.026.0.01.044. | St. Louis encephalitis virus [M16614] (SLEV) |
| | 00.026.0.01.049. | Usutu virus (USUV) |
| | 00.026.0.01.049.04.204.001. | SAAR-1776 [AF013412] |
| | 00.026.0.01.051. | West Nile virus (WNV) |
| | 00.026.0.01.051.04.204.001. | 33/G8; 34/F6 [M12294] |
| | 00.026.0.01.051.04.204.005. | Kunjin virus [D00246] (KUNV) |
| | 00.026.0.01.052. | Yaounde virus (YAOV) |
| | 00.026.0.01.052.03.204.001. | DakArY 276 [AF013413] (YAOV) |
| Kokobera virus group | 00.026.0.01.024. | Kokobera virus (KOKV) |
| | 00.026.0.01.024.04.204.001. | AusMRM 281 [AF013383] (KOKV) |
| | 00.026.0.01.024.04.204.008. | Stratford virus [AF013407] (STRV) |
| Ntaya virus group | 00.026.0.01.004. | Bagaza virus (BAGV) |
| | 00.026.0.01.004.06.206.001. | DakAr B209 [AF013363] (BAGV) |
| | 00.026.0.01.017. | Ilheus virus [AF013376] (ILHV) |
| | 00.026.0.81.003.02.200.001. | Rocio virus [AF013397] (ROCV) |
| | 00.026.0.01.018. | Israel turkey meningoencephalomyelitis |
| | | virus [AF013377] (ITV) |
| | 00.026.0.01.033. | Ntaya virus [AF013392] (NTAV) |
| | 00.026.0.01.045. | Tembusu virus [AF013408] (TMUV) |
| Spondweni virus group | 00.026.0.01.055. | Zika virus (ZIKV) |
| | 00.026.0.01.055.03.200.001. | MR-766 [AF013415] (ZIKAV) |
| | 00.026.0.01.055.03.200.002. | Spondweni virus [AF013406] (SPOV) |
| Yellow fever virus group | 00.026.0.01.005. | Banzi virus (BANV) |
| | 00.026.0.01.005.07.207.001. | SAH 336 [L40951] |
| | 00.026.0.01.007. | Bouboui virus (BOUV) |
| | 00.026.0.01.007.07.207.001. | DakAr B490 [AF013364] |
| | 00.026.0.01.014. | Edge Hill virus (EHV) |
| | 00.026.0.01.014.07.207.001. | Aus C-281 [AF013372] |
| | 00.026.0.01.020. | Jugra virus (JUGV) |
| | 00.026.0.01.020.02.002.001. | P9-314 [AF013378] |
| | 00.026.0.01.039.03.003.001. | Dak An D4600 [AF013400] |
| | 00.026.0.01.039. | Saboya virus (SABV) |
| | 00.026.0.01.039.03.004. | Potiskum virus (POTV) |
| | 00.026.0.01.039.03.004.002. | IBAN 10069 [AF013395] |
| | 00.026.0.01.043. | Sepik virus (SEPV) |
| | 00.026.0.01.043.02.002.001. | MK7148 [AF013404] |
| | 00.026.0.01.048. | Uganda S virus (UGSV) |
| | 00.026.0.01.050. | Wesselsbron virus (WESSV) |
| | 00.026.0.01.053. | Yellow fever virus (YFV) |
| | 00.026.0.01.053.01.201.002. | 17D (vaccine strain) [X03700] |
| | 00.026.0.01.053.01.201.004. | Pasteur 17D-204 (vaccine strain) [X15062] |
| | 00.026.0.01.053.01.201.003. | strain 1899/81 |

3. Viruses with no known arthopod vector

| | | |
|---|---|---|
| Entebbe virus group | 00.026.0.01.015. | Entebbe bat virus (ENTV) |
| | 00.026.0.01.015.03.003.001. | UgIL-30 [AF013373] |
| | 00.026.0.81.015.03.004. | Sokoluk virus (SOKV) |
| | 00.026.0.81.015.03.004.001. | LEIV-400K [AF013405] |
| | 00.026.0.01.054. | Yokose virus (YOKV) |
| | 00.026.0.01.054.06.206.001. | Oita 36 [AB114858] |
| Modoc virus group | 00.026.0.01.002. | Apoi virus [AF013361] (APOIV) |
| | 00.026.0.01.011. | Cowbone Ridge virus (CRV) |
| | 00.026.0.01.011.09.009.001. | W-10986 [AF013370] |
| | 00.026.0.01.021. | Jutiapa virus (JUTV) |
| | 00.026.0.01.021.09.009.001. | JG-128 [AF013379] |
| | 00.026.0.01.030. | Modoc virus (MODV) |
| | 00.026.0.01.030.09.009.001. | M544 [AF013387] |
| | 00.026.0.01.040. | Sal Vieja virus (SVV) |
| | 00.026.0.01.040.09.009.001. | 38TWM-106 [AF013401] |
| | 00.026.0.01.041. | San Perlita virus (SPV) |
| | 00.026.0.01.041.09.009.001. | 71V-1251 [AF013402] |
| Rio Bravo virus group | 00.026.0.01.008. | Bukalasa bat virus (BBV) |
| | 00.026.0.01.008.03.003.001. | UGBP-111 [AF013365] |
| | 00.026.0.01.010. | Carey Island virus (CIV) |
| | 00.026.0.01.010.02.001.001. | P70-1215 [AF013368] |
| | 00.026.0.01.012. | Dakar bat virus (DBV) |
| | 00.026.0.01.012.03.003.001. | 209 [AF013371] |
| | 00.026.0.01.031. | Montana myotis leukoencephalitis virus (MMLV) |
| | 00.026.0.01.031.03.001.001. | 40649 [AF013388] |
| | 00.026.0.01.035. | Phnom Penh bat virus (PPBV) |
| | 00.026.0.01.035.02.001.001. | CAMA-38D [AF013394] |
| | 00.026.0.01.035.02.002. | Batu Cave virus (BCV) |
| | 00.026.0.01.035.02.002.001. | P70-1459 [AF013369] |
| | 00.026.0.01.037. | Rio Bravo virus (RBV) |
| | 00.026.0.01.037.03.003.001. | M-64 [AF013396] |
| Unassigned Members in the Genus | 00.026.0.81.056. | Tamana bat virus (TABV) |
| | 00.026.0.81.057. | Cell fusing agent virus [M91671] (CFAV) |
| Genus | 00.026.0.02. | Pestivirus |
| Type Species | 00.026.0.02.001. | Bovine viral diarrhea virus 1 (BVDV) |

List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, their serotypes, strains and isolates | 00.026.0.02.002. | Border disease virus (sheep) (BDV) |
| | 00.026.0.02.002.00.001.001. | BD31 [U70263] |
| | 00.026.0.02.002.00.001.002. | X818 [AF037405] |
| | 00.026.0.02.003. | Bovine viral diarrhea virus 1 (BVDV-1) |
| | 00.026.0.02.003.00.001.001. | NADL [M31182] |
| | 00.026.0.02.003.00.001.002. | Osloss [M96687] |
| | 00.026.0.02.003.00.001.003. | SD-1 [M96751] |
| | 00.026.0.02.003.00.001.004. | CP7 [U63479] |
| | 00.026.0.02.004. | Bovine viral diarrhea virus 2 (BVDV-2) |
| | 00.026.0.02.004.00.001.001. | strain 890 [U18059] |
| | 00.026.0.02.004.00.001.002. | C413 [AF002227] |
| | 00.026.0.02.005. | Classical swine fever virus (CSFV) |
| | 00.026.0.02.005.00.001.001. | Alfort/187 [X87939] |
| | 00.026.0.02.005.00.001.002. | Alfort-Tübingen [J04358] |
| | 00.026.0.02.005.00.001.003. | Brescia [M31768] |
| | 00.026.0.02.005.00.001.004. | C strain [Z46258] |
| | 00.026.0.02.005. | (Hog cholera virus) (HCV) |
| Unassigned Members in the Genus | 00.026.0.82.006. | Pestivirus of giraffe (H138 (Giraffe-1)) |
| Genus | 00.026.0.03. | Hepacivirus |
| Type Species | 00.026.0.03.001. | Hepatitis C virus (HCV) |

List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, their serotypes, strains and isolates | 00.026.0.03.001. | Hepatitis C virus (HCV) |
| | 00.026.0.03.001.01. | HCV genotype 1 |
| | 00.026.0.03.001.01.001. | subtype 1a [M62321] (HCV-1) |
| | 00.026.0.03.001.01.002. | subtype 1b [D90208] (HCV-J) |
| | 00.026.0.03.001.02. | HCV genotype 2 |
| | 00.026.0.03.001.02.001. | subtype 2a [D00944] (HCV-J6) |
| | 00.026.0.03.001.02.002. | subtype 2b [D01221] (HCV-J8) |
| | 00.026.0.03.001.03. | HCV genotype 3 |
| | 00.026.0.03.001.03.001. | subtype 3a [D17763] (HCV-NZL1) |
| | 00.026.0.03.001.03.010. | subtype 10a [D63821] (HCV-JK049) |
| | 00.026.0.03.001.04. | HCV genotype 4 |
| | 00.026.0.03.001.04.001. | subtype 4a [Y11604] (HCV-ED43) |
| | 00.026.0.03.001.05. | HCV genotype 5 |
| | 00.026.0.03.001.05.001. | subtype 5a [Y13184] (HCV-EVH1480) |
| | 00.026.0.03.001.06. | HCV genotype 6 |
| | 00.026.0.03.001.06.001. | subtype 6a [Y12083] (HCV-EUHK2) |
| | 00.026.0.03.001.06.011. | subtype 11a [D63822] (HCV-JK046) |
| Unassigned Members in the Genus | 00.026.0.83.002. | GB virus B [U22304] (GBV-B) |
| Unassigned Viruses in the Family | 00.026.0.00.001. | GB virus A [U22303] (GBV-A) |
| | 00.026.0.00.002. | GB virus B [U22304] (GBV-B) |
| No Classification Details available | 00.026.0.84.002. | GBV-A-like agents [U94421] (GBV-A-like agents) |
| | 00.026.0.06.001. | GB virus C [U36380] (GBV-C) |
| | 00.026.0.06.002. | Hepatitis G virus [U44402] (HGV-1) |
| | 00.026.0.06.001.00.000.001. | GB virus C troglodytes [AF070476] (GBV-C) |
| | 00.026.0.06.002.00.000.001. | HGV-Iowan [AF121950] (HGV-Iowan) |

TABLE 6
(from the ICTVdB Index of Viruses on the worldwide web at
ncbi.nlm.nih.gov/ICTVdb/Ictv/fs_picor.htm)

| | | |
|---|---|---|
| Family | 00.052. | Picornaviridae |
| Taxonomic Structure of the Family | | |
| Family | 00.052. | Picornaviridae |
| Genus | 00.052.0.01. | Enterovirus |
| Genus | 00.052.0.02. | Rhinovirus |
| Genus | 00.052.0.04. | Cardiovirus |
| Genus | 00.052.0.05. | Aphthovirus |
| Genus | 00.052.0.03. | Hepatovirus |
| Genus | 00.052.0.06. | Parechovirus |
| Genus | 00.052.0.07. | Erbovirus |
| Genus | 00.052.0.08. | Kobuvirus |
| Genus | 00.052.0.09. | Teschovirus |
| Genus | 00.052.0.01. | Enterovirus |
| Type Species | 00.052.0.01.001. | Poliovirus (PV) |

http://rhino.bocklabs.wisc.edu/cgi-bin/virusworld/virustable.pl?virusdata=p1m%2C+Polio+Virus+Type+1+Mahoney%2C+2PLV
List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, their serotypes, strains and isolates | 00.052.0.01.002. | Bovine enterovirus (BEV) |
| | 00.052.0.01.002.00.001. | Bovine enterovirus 1 [D00214] (BEV-1) |
| | 00.052.0.01.002.00.002. | Bovine enterovirus 2 [X79369] (BEV-2) |
| | 00.052.0.01.003. | Human enterovirus A (HEV-A) |
| | 00.052.0.01.003.01.002. | Human coxsackievirus A2 [L28146] (CV-A2) |
| | 00.052.0.01.003.01.002. | Human coxsackievirus A2 [X87585] (CV-A2) |
| | 00.052.0.01.003.01.003. | Human coxsackievirus A3 [X87586] (CV-A3) |
| | 00.052.0.01.003.01.005. | Human coxsackievirus A5 [X87588] (CV-A5) |
| | 00.052.0.01.003.01.007. | Human coxsackievirus A7 [X87589] (CV-A7) |
| | 00.052.0.01.003.01.008. | Human coxsackievirus A8 [X87590] (CV-A8) |
| | 00.052.0.01.003.01.010. | Human coxsackievirus A10 [X87591] (CV-A10) |
| | 00.052.0.01.003.01.012. | Human coxsackievirus A12 [X87593] (CV-A12) |
| | 00.052.0.01.003.01.014. | Human coxsackievirus A14 [X87595] (CV-A14) |
| | 00.052.0.01.003.01.016. | Human coxsackievirus A16 [U05876] (CV-A16) |
| | 00.052.0.01.003.00.071. | Human enterovirus 71 [U22521] (HEV71) |
| | 00.052.0.01.004. | Human enterovirus B (HEV-B) |
| | 00.052.0.01.004.02.001. | Human coxsackievirus B1 [M16560] (CV-B1) |
| | 00.052.0.01.004.02.002. | Human coxsackievirus B2 [AF081485] (CV-B2) |
| | 00.052.0.01.004.02.003. | Human coxsackievirus B3 [M88483] (CV-B3) |
| | 00.052.0.01.004.02.004. | Human coxsackievirus B4 [X05690] (CV-B4) |
| | 00.052.0.01.004.02.005. | Human coxsackievirus B5 [X67706] (CV-B5) |
| (including Swine vesicular disease virus) | 00.052.0.01.004.02.005. | (Swine vesicular disease virus) [D00435] (CV-B5) |
| | 00.052.0.01.004.02.006. | Human coxsackievirus B6 [AF039205] (CV-B6) |
| | 00.052.0.01.004.01.009. | Human coxsackievirus A9 [D00627] (CV-A9) |
| | 00.052.0.01.004.03.001. | Human echovirus 1 [X89531] (EV-1) |
| | 00.052.0.01.004.03.002. | Human echovirus 2 [X89532] (EV-2) |
| | 00.052.0.01.004.03.003. | Human echovirus 3 [X89533] (EV-3) |
| | 00.052.0.01.004.03.004. | Human echovirus 4 [X89534] (EV-4) |
| | 00.052.0.01.004.03.005. | Human echovirus 5 [X89535] (EV-5) |
| | 00.052.0.01.004.03.006. | Human echovirus 6 [U16283] (EV-6) |
| | 00.052.0.01.004.03.007. | Human echovirus 7 [X89538] (EV-7) |
| | 00.052.0.01.004.03.009. | Human echovirus 9 [X84981] (EV-9) |
| | 00.052.0.01.004.03.009. | Human echovirus 9 [X92886] (EV-9) |
| | 00.052.0.01.004.03.011. | Human echovirus 11 [X80059] (EV-11) |
| | 00.052.0.01.004.03.012. | Human echovirus 12 [X79047] (EV-12) |
| | 00.052.0.01.004.03.013. | Human echovirus 13 [X89542] (EV-13) |
| | 00.052.0.01.004.03.014. | Human echovirus 14 [X89543] (EV-14) |
| | 00.052.0.01.004.03.015. | Human echovirus 15 [X89544] (EV-15) |
| | 00.052.0.01.004.03.016. | Human echovirus 16 [X89545] (EV-16) |
| | 00.052.0.01.004.03.017. | Human echovirus 17 [X89546] (EV-17) |
| | 00.052.0.01.004.03.018. | Human echovirus 18 [X89547] (EV-18) |
| | 00.052.0.01.004.03.019. | Human echovirus 19 [X89548] (EV-19) |
| | 00.052.0.01.004.03.020. | Human echovirus 20 [X89549] (EV-20) |
| | 00.052.0.01.004.03.021. | Human echovirus 21 [X89550] (EV-21) |
| | 00.052.0.01.004.03.024. | Human echovirus 24 [X89551] (EV-24) |
| | 00.052.0.01.004.03.025. | Human echovirus 25 [X90722] (EV-25) |
| | 00.052.0.01.004.03.025. | Human echovirus 25 [X89552] (EV-25) |
| | 00.052.0.01.004.03.026. | Human echovirus 26 [X89553] (EV-26) |
| | 00.052.0.01.004.03.027. | Human echovirus 27 [X89554] (EV-27) |
| | 00.052.0.01.004.03.029. | Human echovirus 29 [X89555] (EV-29) |
| | 00.052.0.01.004.03.030. | Human echovirus 30 [X89556] (EV-30) |
| | 00.052.0.01.004.03.031. | Human echovirus 31 [X89557] (EV-31) |
| | 00.052.0.01.004.03.032. | Human echovirus 32 [X89558] (EV-32) |
| | 00.052.0.01.004.03.033. | Human echovirus 33 [X89559] (EV-33) |
| | 00.052.0.01.004.03.069. | Human enterovirus 69 [X87605] (HEV-69) |
| | 00.052.0.01.005. | Human enterovirus C (HEV-C) |
| | 00.052.0.01.005.01.001. | Human coxsackievirus A1 [X87584] (CV-A1) |
| | 00.052.0.01.005.01.011. | Human coxsackievirus A11 [X87592] (CV-A11) |
| | 00.052.0.01.005.01.013. | Human coxsackievirus A13 [X87594] (CV-A13) |
| | 00.052.0.01.005.01.015. | Human coxsackievirus A15 [X87596] (CV-A15) |
| | 00.052.0.01.005.01.017. | Human coxsackievirus A17 [X87597] (CV-A17) |
| | 00.052.0.01.005.01.018. | Human coxsackievirus A18 [X87598] (CV-A18) |
| | 00.052.0.01.005.01.019. | Human coxsackievirus A19 [X87599] (CV-A19) |
| | 00.052.0.01.005.01.020. | Human coxsackievirus A20 [X87600] (CV-A20) |
| | 00.052.0.01.005.01.021. | Human coxsackievirus A21 [D00538] (CV-A21) |
| | 00.052.0.01.005.01.022. | Human coxsackievirus A22 [X87603] (CV-A 22) |
| | 00.052.0.01.005.01.024. | Human coxsackievirus A24 [X90457] (CV-A24) |
| | 00.052.0.01.006. | Human enterovirus D (HEV-D) |
| | 00.052.0.01.006.00.068. | Human enterovirus 68 [X87604] (HEV-68) |
| | 00.052.0.01.006.00.070 | Human enterovirus 70 [D00820] (HEV-70) |
| | 00.052.0.01.010. | Human enterovirus E (HEV-E) |
| | 00.052.0.01.010.00.001. | A-2 plaque virus (proposal withdrawn September 2001) [AF201894] |
| | 00.052.0.01.007. | Poliovirus (PV) |
| | 00.052.0.01.007.00.001. | Human poliovirus 1 (HPV-1) |
| | 00.052.0.01.007.00.001.001. | Mahoney strain [J02281] (HPV-1) |
| | 00.052.0.01.007.00.002. | Human poliovirus 2 (HPV-2) |
| | 00.052.0.01.007.00.002.001. | Lansing strain [M12197] (HPV-1) |
| | 00.052.0.01.007.00.003. | Human poliovirus 3 (HPV-3) |
| | 00.052.0.01.007.00.003.001. | P3/Leon/37 [K01392] (HPV-1) |
| | 00.052.0.01.008. | Porcine enterovirus A (PEV-A) |
| | 00.052.0.01.008.00.008. | Porcine enterovirus 8 [AJ001391] (PEV-8) |
| | 00.052.0.01.009. | Porcine enterovirus B (PEV-B) |
| | 00.052.0.01.009.00.009. | Porcine enterovirus 9 [Y14459] (PEV-9) |
| | 00.052.0.01.009.00.010. | Porcine enterovirus 10 (PEV-10) |

Unassigned Members in the Genus

| | | |
|---|---|---|
| Serotypes not yet assigned to a species | 00.052.0.01.103. | Human coxsackievirus A 4 (CV-A4) |
| | 00.052.0.01.106. | Human coxsackievirus A 6 (CV-A6) |
| | 00.052.0.01.081. | Simian enterovirus 1 (SEV-1) |
| | 00.052.0.01.082. | Simian enterovirus 2 (SEV-2) |
| | 00.052.0.01.083. | Simian enterovirus 3 (SEV-3) |
| | 00.052.0.01.084. | Simian enterovirus 4 (SEV-4) |
| | 00.052.0.01.085. | Simian enterovirus 5 (SEV-5) |
| | 00.052.0.01.086. | Simian enterovirus 6 (SEV-6) |
| | 00.052.0.01.087. | Simian enterovirus 7 (SEV-7) |
| | 00.052.0.01.088. | Simian enterovirus 8 (SEV-8) |
| | 00.052.0.01.089. | Simian enterovirus 9 (SEV-9) |
| | 00.052.0.01.090. | Simian enterovirus 10 (SEV-10) |
| | 00.052.0.01.091. | Simian enterovirus 11 (SEV-11) |
| | 00.052.0.01.092. | Simian enterovirus 12 (SEV-12) |
| | 00.052.0.01.093. | Simian enterovirus 13 (SEV-13) |
| | 00.052.0.01.094. | Simian enterovirus 14 (SEV-14) |
| | 00.052.0.01.095. | Simian enterovirus 15 (SEV-15) |
| | 00.052.0.01.096. | Simian enterovirus 16 (SEV-16) |
| | 00.052.0.01.097. | Simian enterovirus 17 (SEV-17) |
| | 00.052.0.01.098. | Simian enterovirus 18 (SEV-18) |
| | 00.052.0.01.099. | Simian enterovirus N125 (SEV-N125) |
| | 00.052.0.01.100. | Simian enterovirus N203 (SEV-N203) |
| Genus | 00.052.0.02. | Rhinovirus |
| Type Species | 00.052.0.02.001. | Human rhinovirus A (HRV-1A) |

List of Species in the Genus
The ICTVdB virus code and the viruses. Official virus species names are in italics. Tentative virus
species names, alternative names ( ), isolates, strains, serotypes, or subtypes are not italicized.
Virus codes, virus names, arthropod vector and host names { }, serotypes, genome sequence
accession numbers [ ] and assigned abbreviations ( ), are:

| | | |
|---|---|---|
| Species, their serotypes, strains and isolates | 00.052.0.02.001. | Human rhinovirus A (HRV-A) |
| | 00.052.0.02.001.00.001. | Human rhinovirus 1 (HRV-1) |
| | 00.052.0.02.001.00.001.001. | Human rhinovirus 1A (HRV-1A) |
| | 00.052.0.02.001.00.001.002. | Human rhinovirus 1B [D00239] (HRV-1B) |
| | 00.052.0.02.001.00.002. | Human rhinovirus 2 [X02316] (HRV-2) |
| | 00.052.0.02.001.00.007. | Human rhinovirus 7 [Z47564] (HRV-7) |
| | 00.052.0.02.001.00.009. | Human rhinovirus 9 (HRV-9) |
| | 00.052.0.02.001.00.011. | Human rhinovirus 11 [Z47565] (HRV-11) |
| | 00.052.0.02.001.00.015. | Human rhinovirus 15 (HRV-15) |
| | 00.052.0.02.001.00.016. | Human rhinovirus 16 [L24917] (HRV-16) |
| | 00.052.0.02.001.00.021. | Human rhinovirus 21 [Z47566] (HRV-21) |
| | 00.052.0.02.001.00.029. | Human rhinovirus 29 [Z47567] (HRV-29) |
| | 00.052.0.02.001.00.036. | Human rhinovirus 36 [Z49123] (HRV-36) |
| | 00.052.0.02.001.00.039. | Human rhinovirus 39 (HRV-39) |
| | 00.052.0.02.001.00.049. | Human rhinovirus 49 [Z47568] (HRV-49) |
| | 00.052.0.02.001.00.050. | Human rhinovirus 50 [Z47569] (HRV-50) |
| | 00.052.0.02.001.00.058. | Human rhinovirus 58 [Z47570] (HRV-58) |
| | 00.052.0.02.001.00.062. | Human rhinovirus 62 [Z47571] (HRV-62) |
| | 00.052.0.02.001.00.065. | Human rhinovirus 65 [Z47572] (HRV-65) |
| | 00.052.0.02.001.00.085. | Human rhinovirus 85 (HRV-85) |
| | 00.052.0.02.001.00.089. | Human rhinovirus 89 [M16248] (HRV-89) |
| | 00.052.0.02.002. | Human rhinovirus B (HRV-B) |
| | 00.052.0.02.002.00.003. | Human rhinovirus 3 [U60874] (HRV-3) |
| | 00.052.0.02.002.00.014. | Human rhinovirus 14 [K02121] (HRV-14) |
| | 00.052.0.02.002.00.014. | Human rhinovirus 14 [K01087] (HRV-14) |
| | 00.052.0.02.002.00.014. | Human rhinovirus 14 [L05355] (HRV-14) |
| | 00.052.0.02.002.00.072. | Human rhinovirus 72 [Z47574] (HRV-72) |

Unassigned Members in the Genus

| | | |
|---|---|---|
| Serotypes not yet assigned to a species | 00.052.0.02.000.00.301. | Bovine rhinovirus 1 (BRV-1) |
| | 00.052.0.02.000.00.302. | Bovine rhinovirus 2 (BRV-2) |
| | 00.052.0.02.000.00.303. | Bovine rhinovirus 3 (BRV-3) |
| | 00.052.0.02.002.00.004. | Human rhinovirus 4 (HRV-4) |
| | 00.052.0.02.002.00.005. | Human rhinovirus 5 (HRV-5) |
| | 00.052.0.02.002.00.006. | Human rhinovirus 6 (HRV-6) |
| | 00.052.0.02.001.00.008. | Human rhinovirus 8 (HRV-8) |
| | 00.052.0.02.001.00.010. | Human rhinovirus 10 (HRV-10) |
| | 00.052.0.02.001.00.012. | Human rhinovirus 12 (HRV-12) |
| | 00.052.0.02.001.00.013. | Human rhinovirus 13 (HRV-13) |
| | 00.052.0.02.002.00.017. | Human rhinovirus 17 (HRV-17) |
| | 00.052.0.02.001.00.018. | Human rhinovirus 18 (HRV-18) |
| | 00.052.0.02.001.00.019. | Human rhinovirus 19 (HRV-19) |
| | 00.052.0.02.000.00.020. | Human rhinovirus 20 (HRV-20) |
| | 00.052.0.02.001.00.022. | Human rhinovirus 22 (HRV-22) |
| | 00.052.0.02.001.00.023. | Human rhinovirus 23 (HRV-23) |
| | 00.052.0.02.001.00.024. | Human rhinovirus 24 (HRV-24) |
| | 00.052.0.02.001.01.025. | Human rhinovirus 25 (HRV-25) |
| | 00.052.0.02.002.00.026. | Human rhinovirus 26 (HRV-26) |
| | 00.052.0.02.002.00.027. | Human rhinovirus 27 (HRV-27) |
| | 00.052.0.02.001.00.028. | Human rhinovirus 28 (HRV-28) |
| | 00.052.0.02.001.00.030. | Human rhinovirus 30 (HRV-30) |
| | 00.052.0.02.001.00.031. | Human rhinovirus 31 [Z29658] (HRV-31) |
| | 00.052.0.02.000.00.032. | Human rhinovirus 32 (HRV-32) |
| | 00.052.0.02.001.00.033. | Human rhinovirus 33 (HRV-33) |
| | 00.052.0.02.001.00.034. | Human rhinovirus 34 (HRV-34) |
| | 00.052.0.02.000.00.035. | Human rhinovirus 35 (HRV-35) |
| | 00.052.0.02.002.00.037. | Human rhinovirus 37 (HRV-37) |
| | 00.052.0.02.001.00.038. | Human rhinovirus 38 (HRV-38) |
| | 00.052.0.02.000.00.040. | Human rhinovirus 40 (HRV-40) |
| | 00.052.0.02.001.00.041. | Human rhinovirus 41 (HRV-41) |
| | 00.052.0.02.002.00.042. | Human rhinovirus 42 (HRV-42) |
| | 00.052.0.02.000.00.043. | Human rhinovirus 43 (HRV-43) |
| | 00.052.0.02.001.00.044. | Human rhinovirus 44 [Z29660] (HRV-44) |
| | 00.052.0.02.001.00.045. | Human rhinovirus 45 (HRV-45) |
| | 00.052.0.02.000.00.046. | Human rhinovirus 46 (HRV-46) |
| | 00.052.0.02.001.00.047. | Human rhinovirus 47 (HRV-47) |
| | 00.052.0.02.002.00.048. | Human rhinovirus 48 (HRV-48) |
| | 00.052.0.02.001.00.051. | Human rhinovirus 51 (HRV-51) |
| | 00.052.0.02.002.00.052. | Human rhinovirus 52 (HRV-52) |
| | 00.052.0.02.001.00.053. | Human rhinovirus 53 (HRV-53) |
| | 00.052.0.02.001.00.054. | Human rhinovirus 54 (HRV-54) |
| | 00.052.0.02.001.00.055. | Human rhinovirus 55 (HRV-55) |
| | 00.052.0.02.001.00.056. | Human rhinovirus 56 (HRV-56) |
| | 00.052.0.02.000.00.057. | Human rhinovirus 57 (HRV-57) |
| | 00.052.0.02.001.00.059. | Human rhinovirus 59 (HRV-59) |
| | 00.052.0.02.001.00.060. | Human rhinovirus 60 (HRV-60) |
| | 00.052.0.02.001.00.061. | Human rhinovirus 61 (HRV-61) |
| | 00.052.0.02.001.00.063. | Human rhinovirus 63 (HRV-63) |
| | 00.052.0.02.001.00.064. | Human rhinovirus 64 (HRV-64) |
| | 00.052.0.02.001.00.066. | Human rhinovirus 66 (HRV-66) |
| | 00.052.0.02.001.00.067. | Human rhinovirus 67 (HRV-67) |
| | 00.052.0.02.001.00.068. | Human rhinovirus 68 (HRV-68) |
| | 00.052.0.02.002.00.069. | Human rhinovirus 69 (HRV-69) |
| | 00.052.0.02.002.00.070. | Human rhinovirus 70 (HRV-70) |
| | 00.052.0.02.001.00.071. | Human rhinovirus 71 (HRV-71) |
| | 00.052.0.02.001.00.073. | Human rhinovirus 73 (HRV-73) |
| | 00.052.0.02.001.00.074. | Human rhinovirus 74 (HRV-74) |
| | 00.052.0.02.001.00.075. | Human rhinovirus 75 (HRV-75) |
| | 00.052.0.02.001.00.076. | Human rhinovirus 76 (HRV-76) |
| | 00.052.0.02.001.00.077. | Human rhinovirus 77 (HRV-77) |
| | 00.052.0.02.001.00.078. | Human rhinovirus 78 (HRV-78) |
| | 00.052.0.02.002.00.079. | Human rhinovirus 79 (HRV-79) |
| | 00.052.0.02.000.00.080. | Human rhinovirus 80 (HRV-80) |
| | 00.052.0.02.000.00.081. | Human rhinovirus 81 (HRV-81) |
| | 00.052.0.02.000.00.082. | Human rhinovirus 82 (HRV-82) |
| | 00.052.0.02.000.00.083. | Human rhinovirus 83 (HRV-83) |
| | 00.052.0.02.000.00.084. | Human rhinovirus 84 (HRV-84) |
| | 00.052.0.02.002.00.086. | Human rhinovirus 86 (HRV-86) |
| | 00.052.0.02.000.00.087. | Human rhinovirus 87 [AF108187] (HRV-87) |
| | 00.052.0.02.000.00.088. | Human rhinovirus 88 (HRV-88) |
| | 00.052.0.02.000.00.090. | Human rhinovirus 90 (HRV-90) |
| | 00.052.0.02.000.00.091. | Human rhinovirus 91 (HRV-91) |
| | 00.052.0.02.000.00.092 | Human rhinovirus 92 (HRV-92) |
| | 00.052.0.02.000.00.093. | Human rhinovirus 93 (HRV-93) |
| | 00.052.0.02.000.00.094. | Human rhinovirus 94 (HRV-94) |
| | 00.052.0.02.000.00.095. | Human rhinovirus 95 (HRV-95) |
| | 00.052.0.02.000.00.096. | Human rhinovirus 96 (HRV-96) |
| | 00.052.0.02.000.00.097. | Human rhinovirus 97 (HRV-97) |
| | 00.052.0.02.000.00.098. | Human rhinovirus 98 (HRV-98) |
| | 00.052.0.02.000.00.099. | Human rhinovirus 99 (HRV-99) |
| | 00.052.0.02.000.00.100. | Human rhinovirus 100 (HRV-100) |
| Genus | 00.052.0.04. | Cardiovirus |
| Type Species | 00.052.0.04.001. | Encephalomyocarditis virus (EMCV) |

List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, their serotypes, strains and isolates | 00.052.0.04.001. | Encephalomyocarditis virus [M81861] (EMCV) |
| | 00.052.0.04.001.00.001.002. | Mengovirus |
| | 00.052.0.04.001.00.001.001. | Columbia SK virus |
| | 00.052.0.04.001.00.001.003. | Maus Elberfield virus |
| | 00.052.0.04.002. | Theilovirus (ThV) |
| | 00.052.0.04.002.00.002.001. | Theiler's murine encephalomyelitis virus [M20562] (TMEV) |
| | 00.052.0.04.002.00.002.002. | Vilyuisk human encephalomyelitis virus [M80888] (VHEV) |
| | 00.052.0.04.002.00.002.002. | Vilyuisk human encephalomyelitis virus [M94868] (VHEV) |
| | 00.052.0.04.002.00.002.003. | Rat encephalomyelitis virus [M80884] (REV) |
| Unassigned Members in the Genus | | |
| None reported. | | |
| Genus | 00.052.0.05. | Aphthovirus |
| Type Species | 00.052.0.05.001. | Foot-and-mouth disease virus (FMDV) |

List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, their serotypes, strains and isolates | 00.052.0.05.002. | Equine rhinitis A virus [L43052] (ERAV) |
| | 00.052.0.05.002. | Equine rhinitis A virus [X96870] |
| | 00.052.0.05.002. | (formerly Equine rhinovirus 1 virus) (ERV-1) |
| | 00.052.0.05.003. | Foot-and-mouth disease virus (FMDV) |
| | 00.052.0.05.003.00.002. | Foot-and-mouth disease virus A [M10975] (FMDV-A) |
| | 00.052.0.05.003.00.002. | Foot-and-mouth disease virus A [L11360] |
| | 00.052.0.05.003.00.004. | Foot-and-mouth disease virus Asia 1 [U01207] (FMDV-Asia1) |
| | 00.052.0.05.003.00.003. | Foot-and-mouth disease virus C [X00130] (FMDV-C) |
| | 00.052.0.05.003.00.003. | Foot-and-mouth disease virus C [J02191] |
| | 00.052.0.05.003.00.001. | Foot-and-mouth disease virus O [M35873] (FMDV-O) |
| | 00.052.0.05.003.00.001. | Foot-and-mouth disease virus O [X00871] |
| | 00.052.0.05.003.00.005. | Foot-and-mouth disease virus SAT 1 [Z98203] (FMDV-SAT1) |
| | 00.052.0.05.003.00.006. | Foot-and-mouth disease virus SAT 2 [AJ251473] (FMDV-SAT2) |
| | 00.052.0.05.003.00.007. | Foot-and-mouth disease virus SAT 3 [M28719] (FMDV-SAT3) |
| Unassigned Members in the Genus | | |
| None reported. | | |
| Genus | 00.052.0.03. | Hepatovirus |
| Type Species | 00.052.0.03.001. | Hepatitis A virus (HAV) |

List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, their serotypes, strains and isolates | 00.052.0.03.001. | Hepatitis A virus (HAV) |
| | 00.052.0.03.001.00.001.001. | Human hepatitis A virus [M14707] (HHAV) |
| | 00.052.0.03.001.00.001.002. | Simian hepatitis A virus [D00924] (SHAV) |
| Unassigned Members in the Genus | 00.052.0.83.003. | Avian encephalomyelitis-like virus [AJ225173] (AEV) |
| Genus | 00.052.0.06. | Parechovirus |
| Type Species | 00.052.0.06.001. | Human parechovirus (HPeV) |

List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, their serotypes, strains and isolates | 00.052.0.06.001. | Human parechovirus (HPeV) |
| | 00.052.0.06.001.00.001. | Human parechovirus type 1 [L02971] (HPeV-1) |
| | was 00.052.0.01.052. | (formerly Human echovirus 22) (EV-22) |
| | 00.052.0.06.001.00.002. | Human parechovirus type 2 [AJ005695] (HPeV-2) |
| | was 00.052.0.01.053. | (formerly Human echovirus 23) (EV-23) |
| | 00.052.0.06.0.003. | Ljungan virus [AF020541] (LjV) |
| | 00.052.0.06.0.003. | (Rodent parechovirus)(RPeV) |
| | was 00.052.0.86.022. | Ljungan virus (LV) |
| Unassigned Members in the Genus | | |
| None reported. | | |
| Genus | 00.052.0.07. | Erbovirus |
| Type Species | 00.052.0.007.001. | Equine rhinitis B virus (ERBV) |

List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, their serotypes, strains and isolates | 00.052.0.07.001. | Equine rhinitis B virus [X96871] (ERBV) |
| | was 00.052.0.00.004. | (formerly Equine rhinovirus 2) (ERV-2) |
| Unassigned Members in the Genus | | |
| None reported. | | |
| Genus | 00.052.0.08. | Kobuvirus |
| Type Species | 00.052.0.08.001. | Aichi virus (AiV) |

List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, their serotypes, strains and isolates | 00.052.0.08.001. | Aichi virus [AB010145] (AiV) |
| Unassigned Members in the Genus | | |
| None reported. | | |
| Genus | 00.052.0.09. | Teschovirus |
| Type Species | 00.052.0.09.001. | Porcine teschovirus 1 (PTV) |

List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| | | |
|---|---|---|
| Species, their serotypes, strains and isolates | 00.052.0.09.001. | Porcine teschovirus 1 [AJ011380] (PTV-1) |
| | was 00.052.0.01.070. | (formerly Porcine enterovirus 1) (PEV-1) |
| | 00.052.0.09.002. | Porcine teschovirus 2 (PTV-2) |
| | was 00.052.0.01.071. | (formerly Porcine enterovirus 2) (PEV-2) |
| | 00.052.0.09.003. | Porcine teschovirus 3 (PTV-3) |
| | was 00.052.0.01.072 | (formerly Porcine enterovirus 3) (PEV-3) |
| | 00.052.0.09.004. | Porcine teschovirus 4 (PTV-4) |
| | was 00.052.0.01.073. | (formerly Porcine enterovirus 4) (PEV-4) |
| | 00.052.0.09.005. | Porcine teschovirus 5 (PTV-5) |
| | was 00.052.0.01.074. | (formerly Porcine enterovirus 5) (PEV-5) |
| | 00.052.0.09.006. | Porcine teschovirus 6 (PTV-6) |
| | was 00.052.0.01.075. | (formerly Porcine enterovirus 6) (PEV-6) |
| | 00.052.0.09.007. | Porcine teschovirus 7 (PTV-7) |
| | was 00.052.0.01.076. | (formerly Porcine enterovirus 7) (PEV-7) |
| | 00.052.0.09.008. | Porcine teschovirus 11 (PTV-11) |
| | was 00.052.0.01.080. | (formerly Porcine enterovirus 11) (PEV-11) |
| | 00.052.0.09.009. | Porcine teschovirus 12 (PTV-12) |
| | | (formerly Porcine enterovirus 12) (PEV-12) |
| | 00.052.0.09.010. | Porcine teschovirus 13 (PTV-13) |
| | | (formerly Porcine enterovirus 13) (PEV-13) |
| Unassigned Members in the Genus | | |
| None reported. | | |
| List of Unassigned Viruses in the Family | 00.052.0.00.010. | Acid-stable equine picornaviruses (EqPV) |
| | 00.052.0.00.011. | Avian entero-like virus 2 (AELV-2) |
| | 00.052.0.00.012. | Avian entero-like virus 3 (AELV-3) |
| | 00.052.0.00.013. | Avian entero-like virus 4 (AELV-4) |
| | 00.052.0.00.034. | Avian nephritis virus 1 (ANV-1) |
| | 00.052.0.00.014. | Avian nephritis virus 2 (ANV-2) |
| | 00.052.0.00.015. | Avian nephritis virus 3 (ANV-3) |
| | 00.052.0.00.016. | Barramundi virus-1+ (BaV) |
| | 00.052.0.00.017. | Cockatoo entero-like virus (CELV) |
| | 00.052.0.00.018. | Duck hepatitis virus 1 (DHV-1) |
| | 00.052.0.00.019. | Duck hepatitis virus 3 (DHV-3) |
| | 00.052.0.00.005. | Equine rhinovirus 3 (ERV-3) |
| | 00.052.0.00.020. | Guineafowl transmissible enteritis virus (GTEV) |
| | 00.052.0.00.021. | Harbour seal picorna-like virus (SPLV) |
| | 00.052.0.86.022. | Ljungan virus** [AF020541] (LV) |
| | 00.052.0.00.023. | Sea-bass virus-1+ (SBV) |
| | 00.052.0.00.024. | Sikhote-Alyn virus (SAV) |
| | 00.052.0.00.025. | Smelt virus-1+ (SmV-1) |
| | 00.052.0.00.026. | Smelt virus-2+ (SmV-2) |
| | 00.052.0.00.027. | Syr-Daria Valley fever virus (SDFV) |
| | 00.052.0.00.028. | Taura syndrome virus of marine penaeid shrimp (TSV) |
| | 00.052.0.00.029. | Turbot virus-1 (TuV-1) |
| | 00.052.0.00.030. | Turkey entero-like virus (TELV) |
| | 00.052.0.00.031. | Turkey hepatitis virus (THV) |
| | 00.052.0.00.032. | Turkey pseudo enterovirus 1 (TPEV-1) |
| | 00.052.0.00.033. | Turkey pseudo enterovirus 2 (TPEV-2) |

What we claim:

1. Use of a compound of formula I or a pharmaceutically acceptable salt, polymorph, solvate, hydrate, metabolite, prodrug or diastereoisomeric form thereof, for manufacture of a medicament for treatment of virus infections and/or diseases caused by virus infections,
wherein said compound of formula I is:

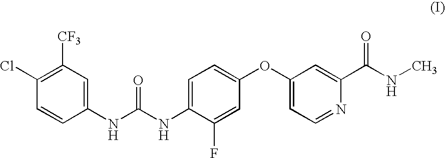

2. Combination comprising at least one compound of formula I as defined in claim 1 and at least one therapeutic agent selected from the group consisting of anti-viral agents, corticosteroids, immunomodulatory agents and known drugs for the therapy of virus infections and/or diseases caused by virus infections.

3. Combination of claim 2 wherein the further therapeutic agent is an anti-viral agent.

4. Combination of claim 2 wherein the further therapeutic agent is lopinavir and/or ritonavir.

5. Combination of claim 2 wherein the further therapeutic agent is selected from the group consisting of lamivudin, parapoxvirus ovis, abacavir, tenofovir disproxil fumarat, emtricitabine, didanosine, stavudine, zidovudine, zalcitabine, efavirenz, nivirapine, delaviridine, atazanavir, ritonavir, amprenavir, lopinavir, rironavir, nelfinavir, indinavir, saquinavir, enfuvirtide, etravirine, capravirine and tenofovir.

6. Combination of claim 2 wherein the further therapeutic agent is indinavir, zidovudine, tenofovir, parapoxvirus ovis and/or lamivudin.

7. Combination of claim 2 wherein the further therapeutic agent is lamivudin and/or adevovir dipivoxil.

8. Combination of claim 2 wherein the further therapeutic agent is oseltamvir and/or zanamivir.

9. Combination of claim 2 wherein the further therapeutic agent is selected from the group consisting of acyclovir, valacyclovir, peniciclovir, famicilovir, foscarnet, brivudin, ganciclovir and cidofovir.

10. Combination of claim 2 wherein the further therapeutic agent is selected from the group consisting of interferon, imiquimod, resiquimod, podophyllin, bleomycin and retinoid.

11. Combination of claim 2 wherein the further therapeutic agent is selected from the group consisting of interferon-β, interferon alfacon-1, interferon-α and pegylated interferon-α.

12. Combination of claim 2 wherein the further therapeutic agent is selected from the group consisting of cidofovir, interferon-β, interferon alfacon-1, interferon-α and pegylated interferon-α.

13. Combination of claim 2 wherein the further therapeutic agent is selected from the group consisting of ribavirin, interferon-β, interferon alfacon-1, interferon-α and pegylated interferon-α.

14. Combination of claim 12 wherein the further therapeutic agent is selected from the group consisting of ruprintrivir (AG 7088), 3C protease inhibitors, pirodavir, pleconaril, soluble ICAM-1, parapoxvirus ovis, interferon-β, interferon alfacon-1, interferon-α and pegylated interferon-α.

15. Combination of any of claims 2 to 14 for the therapy of SARS-CoV, SARS, HBV, HCV, HIV, influenza, Herpesviridae, Paporaviridae, papilloma, Reoviridae, Astroviridae, Bunyaviridae, Filoviridae, Arenaviridae, Rhabdoviridae, Togaviridae, Paramyxoviridae, Poxyiridae, Flaviviridae, Picornaviridae virus infections or unclassified prions and/or diseases caused by said virus infections.

16. Use of a combination of any of claims 2 to 15 for manufacture of a medicament for treatment of virus infections and/or diseases caused by virus infections.

17. The use of any of claims 1 and 16 for the treatment of SARS-CoV, SARS, HBV, HCV, HIV, influenza, Herpesviridae, Paporaviridae, papilloma, Reoviridae, Astroviridae, Bunyaviridae, Filoviridae, Arenaviridae, Rhabdoviridae, Togaviridae, Paramyxoviridae, Poxyiridae, Flaviviridae, Picornaviridae virus infections or unclassified prions and/or diseases caused by said virus infections.

18. The use of any of claims 1 and 16 for the treatment of human herpes simplex viruses, human varizella zoster virus, cytomegalovirus, roseolovirus, Epstein-Barr virus, equine viruses, Aujeszky's virus, suid virus, apish herpesviruses, cercophitecinem herpesviruses, ateline herpesvirus, bovine herpesviruses, feline herpesvirus, canine herpes virus infections and/or diseases caused by such virus infections.

19. The use of any of claims 1 and 16 for the treatment of herpesencephalitis and/or infections of the lymphatic system of the outer genitalia, the lips, the brian and/or the peripheral nerves.

20. The use of any of claims 1 and 16 for the treatment of papillomas, warts and/or neoplasm of the dermis caused by such infections.

21. The use of any of claims 1 and 16 for the treatment of infections by human rotavirus, astrovirus, bunyamweravirus, California encephalitis virus, Hantaan virus, LaCrosse virus, Muerto Canyon virus, Rift Valley Fever virus, sandfly fever virus, tahyna virus, ebola virus, Marburg virus, Junin virus, Lassa virus, lymphotropic choriomeningitis virus, Machupo virus, hydrophobia virus, Duvenhage virus, Mokola virus, vesicular stomatitis virus, Chikungunya virus, Eastern Equine Encephalitis virus, Mayaro virus, O'nyong-nyong virus, ross fever virus, roseola virus, other Equine Encephalitis viruses, measles virus, mumps virus, parainfluenza virus or prions causing Jakob-Creutzfeld disease, BSE or Kuru and its different variants.

22. The use of any of claims 1 and 16 for the treatment of avipoxvirus, capripoxvirus, lepripoxvirus, suipoxvirus, parapoxvirus, molluscipoxvirus, orthopoxvirus infections and/or diseases caused by such virus infections.

23. The use of any of claims 1 and 16 for the treatment of pox and/or Molluscum contagiosum.

24. The use of any of claims 1 and 16 for the treatment of flavivirus, pestivirus infections and/or diseases caused by such virus infections.

25. The use of any of claims 1 and 16 for the treatment of encephalitis and/or encephalomyelitis.

26. The use of any of claims 1 and 16 for the treatment of enterovirus, cardiovirus, rhinovirus, aphtovirus infections and/or diseases caused by such virus infections.

27. The use of any of claims 1 and 16 for the treatment of aseptic meningitis, poliomyelitis, herpangina, pleurodynia (Bornholm disease), myositis, rhabdomyolysis, diabetes type I, summer fever and/or myocarditis.

28. Pharmaceutical composition comprising a combination as defined in any of claims 2 to 15.

29. Pharmaceutical composition of claim 28 for the treatment of virus infections and/or diseases caused by virus infections.

30. Pharmaceutical composition of claim 25 for the treatment of SARS-CoV, SARS, HCV, HIV, influenza, Herpesviridae, Paporaviridae, papilloma, Reoviridae, Astroviridae, Bunyaviridae, Filoviridae, Arenaviridae, Rhabdoviridae, Togaviridae, Paramyxoviridae, Poxyiridae, Flaviviridae, Picornaviridae virus infections or unclassified prions and/or diseases caused by said virus infections.

31. A method for treating virus infections and/or diseases caused by virus infections in a subject in need thereof comprising administering effective amounts of a compound of formula I or a pharmaceutically acceptable salt, polymorph, solvate, hydrate, metabolite, prodrug or diastereoisomeric form thereof
wherein said compound of formula I is:

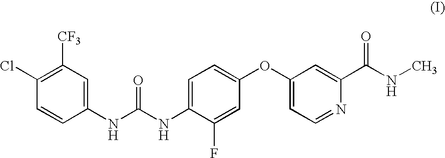

32. The method of claim 31 wherein the compound of formula I is combined with at least one therapeutic agent selected from the group consisting of anti-viral agents, corticosteroids, immunomodulatory agents and known drugs for the therapy of virus infections and/or diseases caused by virus infections.

33. The method of any of claims 31 to 32 for the treatment of SARS-CoV, SARS, HBV, HCV, HIV, influenza, Herpesviridae, Paporaviridae, papilloma, Reoviridae, Astroviridae, Bunyaviridae, Filoviridae, Arenaviridae, Rhabdoviridae, Togaviridae, Paramyxoviridae, Poxyiridae, Flaviviridae, Picornaviridae virus infections or unclassified prions and/or diseases caused by said virus infections.

34. Kit which comprises in separate containers in a single package in one container an effective amount of a compound of formula I as defined in claim 1, in a pharmaceutically acceptable carrier, and in a second container an effective amount of a further therapeutic agent as defined in any of claims 2 to 15, in a pharmaceutically acceptable carrier.
